(19) United States
(12) Patent Application Publication
(10) Pub. No.: US 2001/0031280 A1
(43) Pub. Date: Oct. 18, 2001

(54) Composition comprising at least one hetero polymer and at least one pasty fatty substance and methods for use

(76) Inventors: Veronique Ferrari, Maisons-Alfort (FR); Veronique Jacques, L'Hay Les Roses (FR)

Correspondence Address: FINNEGAN, HENDERSON, FARABOW,, GARRETT & DUNNER, L.L.P., 1300 I Street, N.W., Washington, DC, 20005-3315, US

(21) Appl. No.: 09/749,036

(22) Filed: Dec. 28, 2000

(30) Foreign Application Priority Data
Dec. 28, 1999 (FR) 99 16588
Jan. 25, 2000 (FR) 00 01004

(51) Int. Cl. A61K 7/021; A61K 31/74

(52) U.S. Cl. 424/486; 424/78.26; 424/78.27

(57) ABSTRACT

A physiologically acceptable composition, in particular a cosmetic composition, comprising at least one liquid fatty phase which comprises (i) at least one structuring polymer having a polymer skeleton which comprises at least one hydrocarbon-based repeating unit comprising at least one hetero atom; and (ii) at least one pasty fatty substance.

The present invention relates to a care and/or treatment and/or make-up composition for the skin, including the scalp, and/or for the lips of human beings, and/or for other keratin materials, such as keratinous fibers, containing at least one liquid fatty phase, structured with at least one specific polymer containing at least one hetero atom and at least one pasty fatty substance. This composition is stable over time and can be in the form of a make-up stick, such as lipstick, whose application produces a glossy, migration-resistant deposit, such as a layer, which shows good staying power or long-wearing properties.

It is common to find a structured, i.e., gelled and/or rigidified, liquid fatty phase in cosmetic or dermatological products; this is especially the case in solid compositions such as deodorants, lip balms, lipsticks, concealer products, eyeshadows and cast foundations. This structuring may be obtained with the aid of waxes and/or fillers. Unfortunately, these waxes and fillers may have a tendency to make the composition matte, which may not always be desirable, in particular for a lipstick or an eyeshadow; specifically, consumers are always on the lookout for a lipstick in stick form which can deposit a film with good staying power or long wearing properties but which is also increasingly glossy.

For the purposes of the invention, the expression “liquid fatty phase” means a fatty phase which is liquid at room temperature (25° C.) and atmospheric pressure (760 mmHg), and comprises at least one (as used throughout herein, the expression “at least one” means one or more and thus includes individual components as well as mixtures/combinations) fatty substance that is liquid at room temperature, also referred to as an oil, that is generally mutually compatible. The expression “fatty substance,” such as liquid fatty substance, means a non-aqueous medium which is immiscible in all proportions with water, for example, a hydrocarbon-based compound comprising at least one carbon chain containing at least 5 carbon atoms and possibly comprising at least one polar group chosen from carboxylic acid, hydroxyl, polyol, amine, amide, phosphoric acid, phosphate, ester, ether, urea, carbamate, thiol, thioether and thioester, a silicone compound optionally comprising carbon chains at the end or pendant, these chains optionally being substituted with at least one group chosen from fluoro, perfluoro, (poly)amino acid, ether, hydroxyl, amino, acid and ester groups; or a fluoro or perfluoro compound such as fluorohydrocarbons or perfluorohydrocarbons containing at least 5 carbon atoms, possibly comprising at least one hetero atom chosen from N, O, S and P and optionally comprising at least one polar function chosen from ether, ester, amine, acid, carbamate, urea, thiol and hydroxyl groups.

The structuring of the liquid fatty phase makes it possible, in addition to obtaining a product in the form of a stick or tube, to limit the exudation of the fatty phase from the solid compositions, especially in hot and humid regions, and to limit, after deposition on the skin or the lips, the migration of this phase in the wrinkles and fine lines, which is particularly sought after for a lipstick, a concealer product or an eyeshadow. Specifically, large migration of the liquid fatty phase, in particular when it is charged with coloring agents, may lead to an unpleasant appearance around the lips and the eyes, which particularly makes the wrinkles and fine lines more prominent. This migration is often mentioned by consumers as being a major defect of conventional lipsticks, concealer products and eye make-ups, such as eyeshadows. The term “migration” means a running of the composition beyond the initial application line.

Gloss of a lipstick or other cosmetic is generally associated with the nature of the liquid fatty phase. Thus, it may be possible to reduce the amount of waxes and/or fillers in the composition in order to increase the gloss of a lipstick, but in that case, the migration of the liquid fatty phase may increase. In other words, the amounts of waxes and of fillers required to prepare a stick of suitable hardness which does not exude at room temperature are a restricting factor on the gloss of the deposit.

Furthermore, care, treatment, and make-up compositions should have good staying power or long wearing properties over time and in particular of the color. Poor staying power is characterized by a color change (turning, fading) or a non-uniform change in the make-up effect over time, generally following an interaction with sebum and/or sweat secreted by the skin, and, for the lips, an interaction with saliva. Specifically, a composition which does not have good staying power or long-wearing properties over time may oblige the user to reapply the make-up regularly. However, consumers nowadays wish to enhance the beauty of their face or body while spending as little time as possible in doing so. Finally, a care or make-up composition should be comfortable to wear, for example, non-desiccating and not tightening.

To overcome at least one of these drawbacks, the inventors have envisaged replacing all or some of the waxes and/or fillers with at least one structuring polymer contains at least one heteroatom and at least one pasty fatty substance for structuring the liquid fatty phase. In particular, the inventors have discovered that loss in gloss of a stick comprising waxes is related to the anisotropic crystal structure of such waxes.

In one aspect, the present invention is drawn to a care and/or make-up and/or treatment composition for the skin and/or the lips of the face and/or for superficial body growths, such as keratinous fibres, such as hair, which may make it possible to overcome at least one of the drawbacks mentioned above. In one embodiment, the invention does not include a deodorant product, which is a body hygiene product.

The inventors have found, surprisingly, that the use of at least one specific polymer and at least one pasty fatty substance may make it possible to obtain a composition in rigid form such as a stick, whose application to the skin or the lips produces a deposit which has at least one noteworthy cosmetic property. For example, the deposit may be at least one of glossy, supple, comfortable, light, “migration-resistant,” and having staying power or long-wearing properties.

Moreover, the composition may be stable over time, may withstand shear during application and may not exude at room temperature. In addition, the structuring of the fatty phase of the composition may produce a product that is easy to handle since it does not run between the fingers, unlike a liquid product.

The term “stable” means a composition which does not exude at room temperature (25° C.) for at least 2 months, such as, for example, for at least 9 months.

In one aspect, the invention is drawn to an anhydrous composition comprising at least one liquid fatty phase which comprises (i) at least one structuring polymer comprising a polymer skeleton which comprises at least one hydrocarbon-based repeating unit comprising at least one hetero atom; and (ii) at least one pasty fatty substance.

The invention applies not only to make-up products for the lips, such as lipsticks, lip glosses and lip pencils, but also to care and/or treatment products for the skin, including the scalp, and for the lips, such as antisun products, for example in stick form for facial skin or the lips, care products for the human face or body, make-up products for the skin, both of the human face and body, such as foundations optionally cast in stick or dish form, concealer products, blushers, make-up removing products, eyeshadows, face powders, transfer tattoos, body hygiene products such as deodorants, e.g., in stick form, shampoos, conditioners and make-up products for the eyes such as eyeliners, eye pencils and mascaras, e.g., in stick form, as well as make-up and care products for superficial body growths, for instance keratinous fibers such as the hair, the eyelashes and the eyebrows.

In one aspect, the present invention is drawn to a structured composition containing at least one liquid fatty phase structured with at least one pasty fatty substance and at least one structuring polymer comprising a polymer skeleton comprising at least one hydrocarbon-based repeating unit comprising at least one hetero atom. In one embodiment, the at least one structuring polymer further comprises at least one terminal fatty chain, optionally functionalized, comprising at least one chain chosen from alkyl and alkenyl chains, such as alkyl and alkenyl chains comprising at least 4 carbons atoms, and further such as alkyl and alkenyl chains comprising from 8 to 120, such as from 12 to 120, and such as from 12 to 68 carbon atoms, bonded to the polymer skeleton via at least one linking group. The at least one structuring polymer may also further comprise at least one pendant fatty chain, optionally functionalized, comprising at least one chain chosen from alkyl and alkenyl chains, such as alkyl and alkenyl chains comprising at least 4 carbons atoms, and further such as alkyl and alkenyl chains comprising from 8 to 120, such as from 12 to 120, and such as from 12 to 68 carbon atoms, bonded to any carbon or hetero atom of the polymer skeleton via at least one linking group. The at least one structuring polymer may comprise both at least one pendant fatty chain and at least one terminal fatty chain as defined above. The at least one liquid fatty phase, the at least one structuring polymer and the at least one pasty fatty substance form a physiologically acceptable medium.

In one embodiment, the at least one structuring polymer has a weight-average molecular mass of less than 100,000. In another embodiment, the at least one structuring polymer has a weight-average molecular mass of less than 50,000.

The composition of the invention can be in the form of a paste, a solid or a more or less viscous cream or an anhydrous, rigid, or flexible gel. It can be a single or multiple emulsion, such as an oil-in-water or water-in-oil emulsion or an oil-in-water-in-oil emulsion, or a water-in-oil-in-water emulsion, or a rigid or soft gel containing an oily continuous phase. For example, the liquid fatty phase can be the continuous phase of the composition. In one embodiment, the composition is in a form cast as a stick or in a dish, for example, in the form of an oily rigid gel, such as an anhydrous gel, e.g., an anhydrous stick. In a further embodiment, the composition is in the form of an opaque or translucent rigid gel (depending on the presence or absence of pigments), and in a specific example, the liquid fatty phase forms the continuous phase. The composition of the invention can, for example, be self-supporting.

The structuring of the liquid fatty phase can be modified according to the nature of the at least one structuring polymer and of the at least one pasty fatty substance used, and may be such that a rigid structure in the form of a tube or stick with mechanical strength is obtained. When these tubes or sticks are colored, they may make it possible, after application, to obtain a uniformly, i.e., homogeneously, colored glossy deposit, such as a layer, which does not migrate, for example, into the wrinkles and fine lines of the skin surrounding, for example, the lips and eyes, and which has good staying power or long-wearing properties, in particular of the color, over time.

The composition of the invention may, for example, be a composition for the skin or the lips, such as a foundation composition, concealer product, eyeshadow or lipstick composition, e.g., in stick form.

In one embodiment, the invention is drawn to a structured composition comprising at least one liquid fatty phase which comprises (i) at least one structuring polymer comprising a polymer skeleton which comprises at least one hydrocarbon-based repeating unit comprising at least one hetero atom; and (ii) at least one pasty fatty substance, wherein said at least one pasty fatty substance is not methyl-12-hydroxystearate (methyl-12-hydroxystearate is a wax melting at 52° C. and is not a pasty fatty substance), PEG-40 hydrogenated castor oil (this PEG-40 compound is liquid at room temperature and thus is not in the form of a paste), hydrogenated castor oil (although derivatives of hydrogenated castor oil may be pasty fatty substances, hydrogenated castor oil has a melting point of 82° C. and thus is not a pasty fatty substance), or octadecene dimethyl methyl octadecyl.

Structuring Polymer

In one embodiment, the at least one structuring polymer in the composition of the invention is a solid that is not deformable at room temperature (25° C.) and atmospheric pressure (760 mmHg). In a further embodiment, the at least one structuring polymer is capable of structuring the composition without opacifying it.

The structuring polymer may have a weight-average molecular mass of less than 100,000, such as less than 50,000. In one embodiment, the weight-average molecular mass may range from 1000 to 30,000, such as from 2000 to 20,000, further such as from 2000 to 10,000, further such as 1,000 to 30,000, further such as 1,000 to 10,000, and even further such as 2,000 to 8,000.

In one embodiment, the structuring polymer is a solid which is nondeformable at ambient temperature (25° C.) and atmospheric pressure (760 mm of Hg). It can, for example, be capable of structuring the composition without rendering it opaque.

As defined above, the at least one structuring polymer of the present invention comprises a polymer skeleton comprising at least one hydrocarbon-based repeating unit comprising at least one hetero atom. In one embodiment, the at least one structuring polymer further comprises at least one terminal fatty chain chosen from alkyl and alkenyl chains, such as of at least 4 atoms, and further such as comprising 8 to 120 carbon atoms, bonded to the polymer skeleton via at least one linking group. The terminal fatty chain may, for example, be functionalized. The at least one structuring polymer may also further comprise at least one pendant fatty chain chosen from alkyl and alkenyl chains, such as of at least 4 atoms, and further such as comprising 8 to 120 carbon atoms, bonded to any carbon or hetero atom of the polymer skeleton via at least one linking group. The pendant fatty chain may, for example, be functionalized. The at least one structuring polymer may comprise both at least one pendant fatty chain and at least one terminal fatty chain as defined above, and one or both types of chains can be functionalized.

In one embodiment, the structuring polymer comprises at least two hydrocarbon-based repeating units. As a further example, the structuring polymer comprises at least three hydrocarbon-based repeating units and as an even further example, the at least three repeating units are identical.

As used herein, “functionalized” means comprising at least one functional group. Non-limiting examples of functional groups include hydroxyl groups, ether groups, oxyalkylene groups, polyoxyalkylene groups, carboxylic acid groups, amine groups, amide groups, halogen containing groups, including fluoro and perfluoro groups, halogen atoms, ester groups, siloxane groups and polysiloxane groups.

For purposes of the invention, the expression “functionalized chain” means, for example, an alkyl chain comprising at least one functional (reactive) group chosen, for example, from those recited above. For example, in one embodiment, the hydrogen atoms of at least one alkyl chain may be substituted at least partially with fluorine atoms.

According to the invention, these chains may be linked directly to the polymer skeleton or via, for example, an ester function, perfluoro group, a carboxylic acid, a hydroxyl group, a polyol, an amine, an amide, a phosphoric acid, a phosphate, an ester, an ether, a urea, a carbamate, a thiol, a thioether or a thioester.

For the purposes of the invention, the term “polymer” means a compound containing at least 2 repeating units, such as, for example, a compound containing at least 3 repeating units, which may be identical.

As used herein, the expression “hydrocarbon-based repeating unit” includes a repeating unit comprising from 2 to 80 carbon atoms, such as, for example, from 2 to 60 carbon atoms. The at least one hydrocarbon-based repeating unit may also comprise oxygen atoms. The hydrocarbon-based repeating unit may be chosen from saturated and unsaturated hydrocarbon-based repeating units which in turn may be chosen from linear hydrocarbon-based repeating units, branched hydrocarbon-based repeating units and cyclic hydrocarbon-based repeating units. The at least one hydrocarbon-based repeating unit may comprise, for example, at least one hetero atom that is part of the polymer skeleton, i.e., not pendant. The at least one hetero atom may be chosen, for example, from nitrogen, sulphur, and phosphorus. For example, the at least one hetero atom may be a nitrogen atom, such as a non-pendant nitrogen atom. In another embodiment, the at least one hydrocarbon-based repeating unit may comprise at least one hetero atom with the proviso that the at least one hetero atom is not nitrogen. In another embodiment, the at least one hetero atom is combined with at least one atom chosen from oxygen and carbon to form a hetero atom group. In one embodiment, the hetero atom group comprises a polar group, such as a carbonyl group.

The at least one repeating unit comprising at least one hetero atom may be chosen, for example, from amide groups, carbamate groups, and urea groups. In one embodiment, the at least one repeating unit comprises amide groups forming a polyamide skeleton. In another embodiment, the at least one repeating unit comprises isocyanate units chosen from carbamate groups and urea groups forming a skeleton chosen from polyurethane skeletons, polyurea skeletons, and polyurethane-polyurea skeletons. The pendant chains, for example, can be linked directly to at least one of the hetero atoms of the polymer skeleton, for example, at least one of the nitrogen atoms of an amide group. In yet another embodiment, the at least one hydrocarbon-based repeating unit may comprise at least one hetero atom group with the proviso that the at least one hetero atom group is not an amide group. In one embodiment, the polymer skeleton comprises at least one repeating unit chosen from silicone units and oxyalkylene units, the at least one repeating unit being between the hydrocarbon-based repeating units. In another embodiment, the silicone unit can form an organopolysiloxane backbone.

In one embodiment, the compositions of the invention comprise at least one structuring polymer with nitrogen atoms, such as amide, urea, or carbamate units, such as amide units, and at least one polar oil.

In one embodiment, in the at least one structuring polymer, the percentage of the total number of fatty chains ranges from 40% to 98% relative to the total number of repeating units and fatty chains, and as a further example, from 50% to 95%. In a further embodiment wherein the polymer skeleton is a polyamide skeleton, in the at least one structuring polymer, the percentage of the total number of fatty chains ranges from 40% to 98% relative to the total number of all amide units and fatty chains, and as a further example, from 50% to 95%.

In one embodiment, the linking group is an ester group and is present in an amount ranging from 15% to 40% of the total number of all ester and hetero atom groups in the at least one structuring polymer, such as from 20% to 35%.

In a further embodiment, the nature and proportion of the at least one hydrocarbon-based repeating unit comprising at least one hetero atom depends on the nature of a liquid fatty phase of the composition and is, for example, similar to the nature of the fatty phase. For example, not to be limited as to theory, the more polar the hydrocarbon-based repeating units containing a hetero atom, and in high proportion, which corresponds to the presence of several hetero atoms, the greater the affinity of the at least one structuring polymer to polar oils. Conversely, the more non-polar, or even apolar, and lesser in proportion the hydrocarbon-based repeating units containing a hetero atom, the greater the affinity of the polymer for apolar oils.

In another embodiment, the invention is drawn to a structured composition containing at least one liquid fatty phase structured with at least one pasty fatty substance and at least one structuring polymer, wherein said at least one structuring polymer is a polyamide comprising a polymer skeleton comprising at least one amide repeating unit and optionally at least one pendant fatty chain and/or at least one terminal chain that are/is optionally functionalized and comprising at least one chain chosen from alkyl and alkenyl chains, such as alkyl and alkenyl chains comprising at least 4 carbons atoms, and further such as alkyl and alkenyl chains comprising from 8 to 120 carbon atoms, such as 12 to 120, and further such as 12 to 68, bonded to at least one of the amide repeating units via at least one linking group. The at least one liquid fatty phase, the at least one structuring polyamide and the at least one pasty fatty substance together form a physiologically acceptable medium.

When the structuring polymer has amide repeating units, the pendant fatty chains may be linked to at least one of the nitrogen atoms in the amide repeating units.

As discussed, the at least one structuring polymer may, for example, be chosen from polyamide polymers. A polyamide polymer may comprise, for example, a polymer skeleton which comprises at least one amide repeating unit, i.e., a polyamide skeleton. In one embodiment, the polyamide skeleton may further comprise at least one terminal fatty chain chosen from alkyl chains, for example, alkyl chains comprising at least four carbon atoms, and alkenyl chains, for example, alkenyl chains comprising at least four carbon atoms, bonded to the at least one polyamide skeleton via at least one linking group, and/or at least one pendant fatty chain chosen from alkyl chains, for example, alkyl chains comprising at least four carbon atoms, and alkenyl chains, for example, alkenyl chains comprising at least four carbon atoms, bonded to the at least one polyamide skeleton via at least one linking group. In one embodiment, the polyamide skeleton may comprise at least one terminal fatty chain chosen from fatty chains comprising 8 to 120 carbon atoms, such as, for example, 12 to 68 carbon atoms, bonded to the at least one polyamide skeleton via at least one linking group and/or at least one pendant fatty chain chosen from fatty chains comprising 8 to 120 carbon atoms, such as, for example, 12 to 68 carbon atoms, bonded to the at least one polyamide skeleton via at least one linking group, such as bonded to any carbon or nitrogen of the polyamide skeleton via said at least one linking group.

In one embodiment, the at least one linking group is chosen from single bonds and urea, urethane, thiourea, thiourethane, thioether, thioester, ester, ether and amine groups. In another embodiment, the linking groups are chosen from ureas, esters, and amines, and as a further example, from esters and amines. The bond is, for example, an ester bond. In one embodiment, these polymers comprise a fatty chain at each end of the polymer skeleton, such as the polyamide skeleton.

In a further embodiment, the polyamide polymers can be chosen from polymers resulting from at least one polycondensation reaction between at least one acid chosen from dicarboxylic acids comprising at least 32 carbon atoms, such as 32 to 44 carbon atoms, and at least one amine chosen from diamines comprising at least 2 carbon atoms, such as from 2 to 36 carbon atoms, and triamines comprising at least 2 carbon atoms, such as from 2 to 36 carbon atoms. The dicarboxylic acids can, for example, be chosen from dimers of at least one fatty acid comprising at least 16 carbon atoms, such as oleic acid, linoleic acid and linolenic acid. The at least one amine can, for example, be chosen from diamines, such as ethylenediamine, hexylenediamine, hexamethylenediamine, phenylenediamine and triamines, such as ethylenetriamine.

The polyamide polymers may also be chosen from polymers comprising at least one terminal carboxylic acid group. The at least one terminal carboxylic acid group can, for example, be esterified with at least one alcohol chosen from monoalcohols comprising at least 4 carbon atoms. For example, the at least one alcohol can be chosen from monoalcohols comprising from 10 to 36 carbon atoms. In a further embodiment, the monoalcohols can comprise from 12 to 24 carbon atoms, such as from 16 to 24 carbon atoms, and for example 18 carbon atoms.

In one embodiment, the at least one polyamide polymer may be chosen from those described in U.S. Pat. No. 5,783,657, the disclosure of which is incorporated herein by reference, which are polyamide polymers of formula (I):

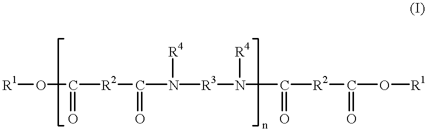

in which:

n is an integer which represents the number of amide units such that the number of ester groups present in said at least one polyamide polymer ranges from 10% to 50% of the total number of all said ester groups and all said amide groups comprised in the at least one polyamide polymer;

R¹, which are identical or different, are each chosen from alkyl groups comprising at least 4 carbon atoms and alkenyl groups comprising at least 4 carbon atoms. In one embodiment, the alkyl group comprises from 4 to 24 carbon atoms and the alkenyl group comprises from 4 to 24 carbon atoms;

R², which are identical or different, are each chosen from C₄ to C₄₂ hydrocarbon-based groups with the proviso that at least 50% of all R² are chosen from C₃₀ to C₄₂ hydrocarbon-based groups;

R³, which are identical or different, are each chosen from organic groups comprising at least one atom, such as two atoms, chosen from carbon atoms, hydrogen atoms, oxygen atoms and nitrogen atoms with the proviso that R³ comprises at least 2 carbon atoms; and

R⁴, which are identical or different, are each chosen from hydrogen atoms, C₁ to C₁₀ alkyl groups and a direct bond to at least one group chosen from R³ and another R⁴ such that when said at least one group is chosen from another R⁴, the nitrogen atom to which both R³ and R⁴ are bonded forms part of a heterocyclic structure defined in part by R⁴—N—R³, with the proviso that at least 50% of all R⁴ are chosen from hydrogen atoms.

In the polymer of formula (I), the terminal fatty chains that are optionally functionalized for the purposes of the invention are terminal chains linked to the last hetero atom, in this case nitrogen, of the polyamide skeleton.

In one embodiment, the ester groups of formula (I), which form part of the terminal and/or pendant fatty chains for the purposes of the invention, are present in an amount ranging from 15% to 40% of the total number of ester and amide groups, such as from 20% to 35%.

In formula (I), in one embodiment, n may be an integer ranging from 1 to 5, for example an integer ranging from 3 to 5. In the present invention, R¹, which are identical or different, can, for example, each be chosen from C₁₂ to C₂₂ alkyl groups, such as from C₁₆ to C₂₂ alkyl groups.

In the present invention, R², which are identical or different, can, for example, each be chosen from C₁₀ to C₄₂ hydrocarbon-based, e.g., alkylene groups. At least 50% of all R², for example at least 75% of all R², which are identical or different, can, for example, each be chosen from groups comprising from 30 to 42 carbon atoms. In the two aforementioned embodiments, the remaining R², which are identical or different, can, for example, each be chosen from C₄ to C₁₈ groups, such as C₄ to C₁₂ groups. In one embodiment, R² can be a C₁₀ to C₄₂ hydrocarbon-based group, e.g., alkylene group, having a structure of polymerized or dimerized fatty acid, the carboxylic acid groups of which have been removed to form the amide.

R³, which can be identical or different, can, for example, each be chosen from C₂ to C₃₆ hydrocarbon-based groups and polyoxyalkylene groups. In another example, R³, which can be identical or different, can each, for example, be chosen from C₂ to C₁₂ hydrocarbon-based groups. In another embodiment, R⁴, which can be identical or different, can each be chosen from hydrogen atoms. As used herein, hydrocarbon-based groups may be chosen from linear, cyclic and branched, and saturated and unsaturated groups. The hydrocarbon-based groups can be chosen from aliphatic and aromatic groups. In one example, the hydrocarbon-based groups are chosen from aliphatic groups. The alkyl and alkylene groups may be chosen from linear, cyclic and branched, and saturated and unsaturated groups.

In general, the pendant and terminal fatty chains may be chosen from linear, cyclic and branched, and saturated and unsaturated groups. The pendant and terminal fatty chains can be chosen from aliphatic and aromatic groups. In one example, the pendant and terminal fatty chains are chosen from aliphatic groups.

According to the invention, the structuring of the liquid fatty phase is obtained with the aid of at least one structuring polymer, such as the at least one polymer of formula (I). The at least one polyamide polymer of formula (I) may, for example, be in the form of a mixture of polymers, and this mixture may also comprise a compound of formula (I) wherein n is equal to zero, i.e., a diester.

Non-limiting examples of an at least one polyamide polymer which may be used in the composition according to the present invention include the commercial products sold by Arizona Chemical under the names Uniclear 80 and Uniclear 100. These are sold, respectively, in the form of an 80% (in terms of active material) gel in a mineral oil and a 100% (in terms of active material) gel. These polymers have a softening point ranging from 88° C. to 94° C., and may be mixtures of copolymers derived from monomers of (i) C₃₆ diacids and (ii) ethylenediamine, and have a weight-average molecular mass of about 6000. Terminal ester groups result from esterification of the remaining acid end groups with at least one alcohol chosen from cetyl alcohol and stearyl alcohol. A mixture of cetyl and stearyl alcohols is sometimes called cetylstearyl alcohol.

Other non-limiting examples of an at least one polyamide polymer which may be used in the compositions according to the present invention include polyamide polymers resulting from the condensation of at least one aliphatic dicarboxylic acid and at least one diamine, the carbonyl and amine groups being condensed via an amide bond. In one embodiment, these polymers contain more than two carbonyl groups and more than two amine groups. Examples of these polyamide polymers are those sold under the brand name Versamid by the companies General Mills Inc. and Henkel Corp. (Versamid 930, 744 or 1655) or by the company Olin Mathieson Chemical Corp. under the brand name Onamid, in particular Onamid S or C. These resins have a weight-average molecular mass ranging from 6000 to 9000. For further information regarding these polyamides, reference may be made to U.S. Pat. Nos. 3,645,705 and 3,148,125, the disclosures of which are hereby incorporated by reference. In one embodiment, Versamid 930 or 744 may be used.

Other examples of polyamides include those sold by the company Arizona Chemical under the references Uni-Rez (2658, 2931, 2970, 2621, 2613, 2624, 2665, 1554, 2623 and 2662) and the product sold under the reference Macromelt 6212 by the company Henkel. For further information regarding these polyamides, reference may be made to U.S. Pat. No. 5,500,209, the disclosure of which is hereby incorporated by reference. Such polyamides display high melt viscosity characteristics. MACROMELT 6212, for example, has a high melt viscosity at 190° C. of 30-40 poise (as measured by a Brookfield Viscometer, Model RVF #3 spindle, 20 RPM).

In a further embodiment, the at least one polyamide polymer may be chosen from polyamide resins from vegetable sources. Polyamide resins from vegetable sources may be chosen from, for example, the polyamide resins of U.S. Pat. Nos. 5,783,657 and 5,998,570, the disclosures of which are herein incorporated by reference.

The at least one structuring polymer in the compositions of the invention may have a softening point greater than 50° C., such as from 65° C. to 190° C., and further such as from 70° C. to 130° C., and even further such as from 80° C. to 105° C. This softening point may be lower than that of structuring polymers used in the art which may facilitate the use of the at least one structuring polymer of the present invention and may limit the degradation of the liquid fatty phase. These polymers may be non waxy polymers.

In one embodiment, due to the presence of at least one chain, the polyamide polymers may be readily soluble in oils (i.e., water-immiscible liquid compounds) and thus may give macroscopically homogeneous compositions even with a high content (at least 25%) of the polyamide polymers, unlike certain polymers of the prior art that do not contain such alkyl or alkenyl chains at the end of the polyamide skeleton. As defined herein, a composition is soluble if it has a solubility of greater than 0.01 g per 100 ml of solution at 25° C.

The at least one structuring polymer may be present in the composition in an amount ranging, for example, from 0.5% to 80% by weight relative to the total weight of the composition, such as for example 2% to 60%, and further, for example, from 5 to 40%. In a further embodiment the at least one structuring polymer may be present in the composition in an amount ranging, for example, from 5% to 25% by weight relative to the total weight of the composition.

In another embodiment of the invention, the present invention is drawn to a structured composition comprising at least one liquid fatty phase structured with at least one structuring polymer comprising a polymer skeleton comprising at least one hydrocarbon-based repeating unit comprising at least one hetero atom, wherein the at least one structuring polymer further comprises at least one terminal fatty chain, optionally functionalized, chosen from alkyl and alkenyl chains, such as alkyl and alkenyl chains having at least four carbon atoms, and further such as alkyl and alkenyl chains comprising from 8 to 120 carbon atoms, bonded to the polymer skeleton via at least one linking group chosen from amides, ureas, and esters, wherein when said at least one linking group is chosen from esters, said at least one terminal fatty chain is chosen from branched alkyl groups. The at least one structuring polymer may also comprise at least one pendant fatty chain, optionally functionalized, chosen from alkyl and alkenyl chains, such as alkyl and alkenyl chains having at least four carbon atoms, and further such as alkyl and alkenyl chains comprising from 8 to 120 carbon atoms, bonded to any carbon or hetero atom of the polymer skeleton via at least one linking group chosen from amides, ureas, and esters, wherein when said at least one linking group is chosen from esters, said at least one terminal fatty chain is chosen from branched alkyl groups. The at least one structuring polymer may comprise both at least one pendant fatty chain and at least one terminal fatty chain as defined above in this paragraph.

Amphiphilic Compound

The at least one structuring polymer can be combined with at least one amphiphilic compound that is liquid and non-volatile at room temperature and has a hydrophilic/lipophilic balance (HLB) value of less than 12, for example ranging from 1 to 8 or from 1 to 7 or from 3 to 5 or from 1 to 5. These amphiphilic compounds may act to reinforce the structuring properties of the at least one structuring polymer, to facilitate the implementation of the at least one structuring polymer and to improve the ability of the stick to be deposited. However, it is possible to obtain a stick with good mechanical and/or thermal properties without including at least one amphiphilic compound.

The at least one amphiphilic compound which can be used in the composition of the invention may, for example, comprise a lipophilic part linked to a polar part, the lipophilic part comprising a carbon-based chain containing at least 8 carbon atoms, for example from 18 to 32 carbon atoms, and further for example from 18 to 28 carbon atoms. The polar part of the at least one amphiphilic compound may, in one embodiment, be the residue of a compound chosen from alcohols and polyols containing from 1 to 12 hydroxyl groups, and polyoxyalkylenes comprising at least 2 oxyalkylene units and containing from 0 to 20 oxypropylene units and/or from 0 to 20 oxyethylene units. For example, the at least one amphiphilic compound may be an ester chosen from the hydroxystearates, oleates and isostearates of glycerol, of sorbitan and of methylglucose, and from branched C₁₂ to C₂₆ fatty alcohols such as octyldodecanol. Among these esters, monoesters and mixtures of mono- and diesters can also be used.

The respective contents of the at least one pasty fatty substance, the at least one structuring polymer and optionally that of at least one amphiphilic compound are chosen according to the desired hardness of the composition and as a function of the specific application envisaged. The respective amounts of polymer, of pasty fatty substance and of amphiphilic compound should be such that they produce a stick which can be worn down. In practice, the amount of the at least one polymer may be chosen from 0.5% to 80% of the total weight of the composition, for example from 2% to 60%, from 5% to 40%, and from 5% to 25%. The amount of at least one amphiphilic compound in practice, if it is present, may be chosen from 0.1% to 35% of the total weight of the composition, for example from 1% to 20%, from 1% to 15%, and from 2% to 15%.

Depending on the intended application, such as a stick, hardness of the composition may also be considered. The hardness of a composition may, for example, be expressed in grams (g). The composition of the present invention may, for example, have a hardness ranging from 20 g to 2000 g, such as from 20 g to 900 g, further such as from 20 g to 600 g, further such as 50 g to 600 g, and further such as 150 g to 450 g.

This hardness is measured in one of two ways. A first test for hardness is according to a method of penetrating a probe into said composition and in particular using a texture analyzer (for example TA-XT2 from Rheo) equipped with an ebonite cylinder of height 25 mm and diameter 8 mm. The hardness measurement is carried out at 20° C. at the center of 5 samples of said composition. The cylinder is introduced into each sample of composition at a pre-speed of 2 mm/s and then at a speed of 0.5 mm/s and finally at a post-speed of 2 mm/s, the total displacement being 1 mm. The recorded hardness value is that of the maximum peak observed. The measurement error is ±50 g.

The second test for hardness is the “cheese wire” method, which involves cutting an 8.1 mm or 12.7 mm in diameter tube of composition and measuring its hardness at 20° C. using a DFGHS 2 tensile testing machine from Indelco-Chatillon Co. at a speed of 100 mm/minute. The hardness value from this method is expressed in grams as the shear force required to cut a stick under the above conditions. According to this method, the hardness of compositions according to the present invention which may be in stick form may, for example, range from 30 g to 300 g, such as from 30 g to 250 g, further such as from 30 g to 200 g, further such as 30 to 150, further such as 30 to 120, and further such as 30 to 50.

The hardness of the composition of the present invention may be such that the compositions are self-supporting and can easily disintegrate to form a satisfactory deposit on a keratin material, such as, for example the skin or lips of human beings. In addition, this hardness may impart good impact strength to the inventive compositions which may be molded or cast, for example, in stick or dish form.

The skilled artisan may choose to evaluate a composition using at least one of the tests for hardness outlined above based on the application envisaged and the hardness desired. If one obtains an acceptable hardness value, in view of the intended application, from at least one of these hardness tests, the composition falls within the scope of the invention.

As is evident, the hardness of the composition according to the invention may, for example, be such that the composition is advantageously self-supporting and can disintegrate easily to form a satisfactory deposit on the skin and/or the lips and/or superficial body growths, such as keratinous fibres. In addition, with this hardness, the composition of the invention may have good impact strength.

According to the invention, the composition in stick form may have the behavior of a deformable, flexible elastic solid, giving suitable elastic softness on application.

Liquid Fatty Phase

The at least one liquid fatty phase, in one embodiment, may comprise at least one oil. The at least one oil, for example, may be chosen from at least one polar oil and at least one apolar oil including hydrocarbon-based liquid oils and oily liquids at room temperature. In one embodiment, the compositions of the invention comprise at least one structuring polymer and at least one polar oil. The polar oils of the invention, for example, may be added to the apolar oils, the apolar oils acting in particular as co-solvent for the polar oils.

In one embodiment, the liquid fatty phase of the composition comprises at least one oil having a group similar to that of the structuring polymer repeating units comprising at least one heteroatom. In particular, for a liquid fatty phase structured by a polymer with a partially silicone-comprising backbone, this fatty phase may comprise a liquid silicone oil or a mixture of liquid silicone oils.

The liquid fatty phase of the composition may contain more than 30%, for example, more than 40%, of liquid oil(s) having a chemical nature close to the chemical nature of the skeleton (hydrocarbon or silicone based) of the structuring polymer, and for example from 50% to 100%. In one embodiment, the liquid fatty phase structured with a polyamide-type skeleton, or polyurea, or polyurethan, or polyurea-urethane-type skeleton contains a high quantity, i.e., greater than 30%, for example greater than 40% relative to the total weight of the liquid fatty phase, or from 50% to 100%, of at least one apolar, such as hydrocarbon-based, oil.

In another embodiment, for a liquid fatty phase structured by a nonpolar polymer comprising hydrocarbon groups, the liquid fatty phase comprises at least one nonpolar liquid oil, such as a hydrocarbon-based oil. For the purposes of the invention, the expression “hydrocarbon-based oil” means an oil comprising carbon and hydrogen atoms, optionally with at least one group chosen from hydroxyl, ester, carboxyl and ether groups.

For a liquid fatty phase structured with a polymer containing a partially silicone-based skeleton, this fatty phase may contain more than 30%, for example, more than 40%, relative to the total weight of the liquid fatty phase and, for example, from 50% to 100%, of at least one silicone-based liquid oil, relative to the total weight of the liquid fatty phase.

For a liquid fatty phase structured with an apolar polymer of the hydrocarbon-based type, this fatty phase may contain more than 30%, for example more than 40% by weight, and, as a further example, from 50% to 100% by weight, of at least one liquid apolar, such as hydrocarbon-based, oil, relative to the total weight of the liquid fatty phase.

For example, the at least one polar oil useful in the invention may be chosen from:

hydrocarbon-based plant oils with a high content of triglycerides comprising esters chosen from fatty acid esters and esters of glycerol in which the fatty acids may have varied chain lengths, such as from C₄ to C₂₄, these chains possibly being chosen from linear and branched, and saturated and unsaturated chains; these oils can be chosen from, for example, wheat germ oil, corn oil, sunflower oil, karite butter, castor oil, sweet almond oil, macadamia oil, apricot oil, soybean oil, cotton oil, alfalfa oil, poppy oil, pumpkinseed oil, sesame oil, cucumber oil, marrow oil, rapeseed oil, avocado oil, hazelnut oil, grape seed oil, blackcurrant seed oil, evening primrose oil, millet oil, barley oil, quinoa oil, olive oil, rye oil, safflower oil, candlenut oil, passion flower oil and musk rose oil; or alternatively caprylic/capric acid triglycerides such as those sold by Stearineries Dubois or those sold under the names Miglyol 810, 812 and 818 by Dynamit Nobel;

synthetic oils or esters of formula R₅COOR₆ in which R₅ is chosen from linear and branched fatty acid residues containing from 1 to 40 carbon atoms, such as from 7 to 30 carbon atoms, and R₆ is chosen from, for example, a hydrocarbon-based chain containing from 1 to 40 carbon atoms, such as from 3 to 40 carbon atoms, on condition that R₅+R₆≧10, such as, for example, purcellin oil (cetostearyl octanoate), isononyl isononanoate, C₁₂-C₁₅ alkyl benzoates, isopropyl myristate, 2-ethylhexyl palmitate, isostearyl isostearate and alkyl or polyalkyl octanoates, decanoates or ricinoleates; hydroxylated esters such as isostearyl lactate and diisostearyl malate; and pentaerythritol esters;

synthetic ethers containing from 10 to 40 carbon atoms;

C₈ to C₂₆ fatty alcohols such as oleyl alcohol; and

C₈ to C₂₆ fatty acids such as oleic acid, linolenic acid and linoleic acid.

The at least one apolar oil according to the invention is chosen from, for example, silicone oils chosen from volatile and non-volatile, linear and cyclic polydimethylsiloxanes (PDMSs) that are liquid at room temperature; polydimethylsiloxanes comprising alkyl or alkoxy groups which are pendant and/or at the end of the silicone chain, the groups each containing from 2 to 24 carbon atoms; phenylsilicones such as phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates; hydrocarbons chosen from linear and branched, volatile and non-volatile hydrocarbons of synthetic and mineral origin, such as volatile liquid paraffins (such as isoparaffins and isododecane) or non-volatile liquid paraffins and derivatives thereof, liquid petrolatum, liquid lanolin, polydecenes, hydrogenated polyisobutene such as Parleam®, and squalane; fluorocarbons chosen from linear and branched, volatile and non-volatile fluorocarbons; and mixtures thereof. The structured oils, for example those structured with polyamides such as those of formula (I) or with polyurethanes, polyureas, polyurea-urethanes, in accordance with the invention, may be, in one embodiment, apolar oils, such as an oil or a mixture of hydrocarbon oils chosen from those of mineral and synthetic origin, chosen from hydrocarbons such as alkanes such as Parleam® oil, isoparaffins including isododecane, and squalane, and mixtures thereof. These oils may, in one embodiment, be combined with at least one phenylsilicone oil.

The at least one liquid fatty phase, in one embodiment, contains at least one non-volatile oil chosen from, for example, hydrocarbon-based oils of mineral, plant and synthetic origin, synthetic esters or ethers, silicone oils and mixtures thereof.

In practice, the total liquid fatty phase can be, for example, present in an amount ranging from 1% to 99% by weight relative to the total weight of the composition, for example from 5% to 95.5%, as a further example from 10% to 80%, and, as an even further example, from 20% to 75%.

For the purposes of the invention, the expression “volatile solvent or oil” means any non-aqueous medium capable of evaporating on contact with the skin or the lips in less than one hour at room temperature and atmospheric pressure. The volatile solvent(s) of the invention is(are) organic solvents, such as volatile cosmetic oils that are liquid at room temperature, having a non-zero vapor pressure, at room temperature and atmospheric pressure, ranging in particular from 10⁻² to 300 mmHg and, for example, greater than 0.3 mmHg. The expression “non-volatile oil” means an oil which remains on the skin or the lips at room temperature and atmospheric pressure for at least several hours, such as those having a vapor pressure of less than 10² mmHg.

According to the invention, these volatile solvents may facilitate the staying power or long wearing properties of the composition on the skin, the lips or superficial body growths, such as keratinous fibers. The solvents can be chosen from hydrocarbon-based solvents, silicone solvents optionally comprising alkyl or alkoxy groups that are pendant or at the end of a silicone chain, and a mixture of these solvents.

The volatile oil(s), in one embodiment, is present in an amount ranging up to 95.5% relative to the total weight of the composition, such as from 2% to 75%, and, as a further example, from 10% to 45%. This amount will be adapted by a person skilled in the art according to the desired staying power or long wearing properties.

The at least one liquid fatty phase of the compositions of the invention may further comprises a dispersion of lipid vesicles. The compositions of the invention may also, for example, be in the form of a fluid anhydrous gel, a rigid anhydrous gel, a fluid simple emulsion, a fluid multiple emulsion, a rigid simple emulsion or a rigid multiple emulsion. The simple emulsion or multiple emulsion may comprise a continuous phase chosen from an aqueous phase optionally containing dispersed lipid vesicles or oil droplets, or a fatty phase optionally containing dispersed lipid vesicles or water droplets. In one embodiment, the composition has a continuous oily phase or fatty phase and is more specifically an anhydrous composition, for example, a stick or dish form. An anhydrous composition is one that has less than 10% water by weight, such as, for example, less than 5% by weight.

Pasty Fatty Substance

The composition according to the invention also comprises at least one fatty compound that is pasty at room temperature. A pasty fatty substance is a viscous product comprising a liquid fraction and a solid fraction. For the purposes of the invention, the expression “pasty fatty substance” includes a fatty substance with a melting point ranging from 20 to 55° C., for example from 25 to 45° C., and/or a viscosity at 40° C. ranging from 0.1 to 40 Pa.s (1 to 400 poises), for example from 0.5 to 25 Pa.s, measured using a Contraves TV or Rheomat 80 viscometer, equipped with a spindle rotating at 60 Hz. A person skilled in the art can select the spindle for measuring the viscosity from the spindles MS-r3 and MS-r4, on the basis of his general knowledge, so as to be able to carry out the measurement of the pasty compound tested.

The melting point values correspond, according to the invention, to the melting peak measured by the “Differential Scanning Calorimetry” method with a rising temperature of 5 or 10° C./min.

According to the present invention, the at least one pasty fatty substance may be chosen from hydrocarbon-based compounds, optionally of polymer type, silicone compounds and fluorinated compounds. In one embodiment, the hydrocarbon-based compounds contain ester groups. Of course, the at least one pasty fatty substance can also be a mixture of hydrocarbon-based and/or silicone and/or fluorinated compounds. In one embodiment, the at least one pasty fatty substance is a mixture of hydrocarbon-based and/or silicone and/or fluorinated compounds, wherein the hydrocarbon-based pasty fatty compounds are present in the composition in an amount greater than either the silicone pasty fatty compounds or the fluorinated pasty fatty compounds. In one embodiment, a mixture of past fatty substances are used in which the pasty fatty substances are predominantly hydrocarbon-based past fatty substances.

Representative pasty fatty substances which may be used in the composition according to the invention include lanolins and lanolin derivatives such as acetylated lanolins or oxypropylenated lanolins or isopropyl lanolate, having a viscosity of from 18 to 21 Pa.s, for instance 19 to 20.5 Pa.s, and/or a melting point of from 30 to 55° C. The at least one pasty fatty substance may also be chosen from esters of fatty acids or of fatty alcohols, such as those containing from 20 to 65 carbon atoms (melting point of about from 20 to 35° C. and/or viscosity at 40° C. ranging from 0.1 to 40 Pa.s), such as triisostearyl citrate or cetyl citrate; arachidyl propionate; polyvinyl laurate; cholesterol esters, such as triglycerides of plant origin, such as hydrogenated plant oils, viscous polyesters such as poly(12-hydroxystearic acid); and silicone pasty fatty substances, such as polydimethylsiloxanes (PDMS) having alkyl or alkoxy pendant chains containing from 8 to 24 carbon atoms, and a melting point of from 20 to 55° C., such as stearyidimethicones (in particular DC2503 and DC25514 from Dow Corning). In one embodiment, the triglycerides of plant origin are chosen from triglycerides of vegetable origin, such as derivatives of hydrogenated castor oil, such as “Thixinr” from Rheox.

The at least one pasty fatty substance may be present in a proportion ranging, for example, from 0.5% to 60% by weight, relative to the total weight of the composition, such as from 2% to 45% by weight, and further such as from 5% to 30% by weight.

Furthermore, it has been found that the composition according to the present invention may make it possible to obtain a film with a homogeneous colouring, optionally in the presence of at least one coloring material, due to a good wetting of the at least one coloring material by the at least one pasty fatty substance. Further, the film obtained may be applied easily and may spread easily on a substrate, such as a keratin material. The film may also exhibit a light texture and may be very comfortable to wear throughout the day.

Additional Additive

The composition of the invention can also comprise any additional additive usually used in the field under consideration, such as cosmetics or dermatology, chosen, for example, antioxidants, essential oils, preservatives, fragrances, fillers (such as Polytrap® from Dow Corning), waxes, neutralizing agents, dispersing agents, fat-soluble polymers, cosmetic or dermatological active agents such as, for example, emollients, moisturizers, vitamins, essential fatty acids, and sunscreens and mixtures thereof. These additives, if present, may each be present in the composition in a proportion of up to 30%, for example from 0.01% to 20%, and, as a further example, from 0.01% to 10% relative to the total weight of the composition.

The composition may also contain at least one cosmetic active agent and/or one dermatological active agent and/or at least one coloring agent.

The composition of the invention can also contain, as an additional additive, an aqueous phase containing water that is optionally thickened with an aqueous-phase thickener or gelled with a hydrophilic gelling agent and optionally water-miscible compounds.

Needless to say, a person skilled in the art will take care to select the optional additional additives and/or the amount thereof such that the advantageous properties of the composition according to the invention are not, or are not substantially, adversely affected by the envisaged addition.

The composition according to the invention can be in the form of a tinted dermatological composition or care composition for keratin materials such as the skin, the lips and/or superficial body growths, such as keratinous fibers, in the form of an antisun composition or body hygiene composition, such as in the form of a deodorant product or make-up-removing product in stick form. It can be used, for example, as a care base for the skin, superficial body growths or the lips, for example, lip balms, for protecting the lips against cold and/or sunlight and/or the wind, or care cream for the skin, the nails or the hair. In one embodiment, the composition is provided in an uncolored form and includes one or more active ingredients.

The composition of the invention may also be in the form of a colored make-up product for the skin, optionally having care or treating properties, such as a foundation, a blusher, a face powder, an eyeshadow, a concealer product, an eyeliner, a make-up product for the body; a make-up product for the lips such as a lipstick; a make-up product for superficial body growths, such as the nails or the eyelashes, for example in the form of a mascara, or for the eyebrows and the hair, for example in the form of a pencil. In one embodiment, the composition exhibits care or treating properties and is in the form of a product for making up the lips.

Needless to say, the composition of the invention should be cosmetically or dermatologically acceptable, i.e., it should contain a non-toxic physiologically acceptable medium and should be able to be applied to the skin, superficial body growths or the lips of human beings. For the purposes of the invention, the expression “cosmetically acceptable” means a composition of pleasant appearance, odor and feel.

Coloring Agent

The present invention may also comprise at least one coloring agent which may be chosen from the lipophilic dyes, hydrophilic dyes, pigments and nacres usually used in cosmetic or dermatological compositions. The at least one coloring agent can generally be present in a proportion of from, for example, 0.01% to 50% relative to the total weight of the composition, further for example from 0.5% to 40%, and, as a further example, from 5% to 30%, such as from 5% to 25%, if it is present. In one embodiment, in the case of a composition in the form of a free or compacted powder, the amount of coloring agent in the form of solid particles that are insoluble in the medium (nacres and/or pigments) may be up to 90% relative to the total weight of the composition.

The liposoluble dyes include, for example, Sudan Red, D&C Red 17, D&C Green 6, β-carotene, soybean oil, Sudan Brown, D&C Yellow 11, D&C Violet 2, D&C Orange 5, quinoline yellow and annatto. They can be present in an amount ranging from 0% to 20% of the weight of the composition, for example from 0.1% to 6% (if present). The water-soluble dyes are, for example, beetroot juice or methylene blue, and can represent, for example, up to 6% of the total weight of the composition.

The pigments may be white or colored, inorganic and/or organic, and coated or uncoated and having a micron size or not. Among the inorganic pigments which may be mentioned are titanium dioxide, optionally surface-treated, zirconium oxide, zinc oxide or cerium oxide, as well as iron oxide, chromium oxide, manganese violet, ultramarine blue, chromium hydrate and ferric blue. Among the organic pigments which may be mentioned are carbon black, pigments of D&C type, and lakes based on cochineal carmine or on barium, strontium, calcium or aluminium. The pigment(s) can be present in an amount ranging, for example, from 0.1% to 50%, further for example from 0.5% to 40%, and, as a further example, from 2% to 30%, such as from 2% to 25% relative to the total weight of the composition, if they are present.

The nacreous pigments may be chosen from white nacreous pigments such as mica coated with titanium oxide or with bismuth oxychloride, colored nacreous pigments such as titanium oxide-coated mica with iron oxides, titanium oxide-coated mica with, for example, ferric blue or chromium oxide, titanium oxide-coated mica with an organic pigment of the type mentioned above, as well as nacreous pigments based on bismuth oxychloride. They can be present in an amount ranging, for example, from 0.1% to 20% relative to the total weight of the composition, further for example from 0. 1% to 15%, if they are present.

Waxes

The composition can optionally contain at least one wax to further limit the exudation of the composition in stick form, although this rigid form can be obtained in the absence of wax. For the purposes of the present invention, a wax is a lipophilic fatty compound that is solid at room temperature (25° C.) and atmospheric pressure (760 mmHg), which undergoes a reversible solid/liquid change of state, having a melting point of greater than 40° C., for example greater than 55° C. and which may be up to 200° C., and having an anisotropic crystal organization in the solid state. In one embodiment, the size of the crystals is such that the crystals diffract and/or scatter light, conferring a more or less opaque cloudy appearance on the composition. By bringing the wax to its melting point, it is possible to make it miscible with oils and to form a microscopically homogeneous mixture, but on returning the temperature of the mixture to room temperature, recrystallization of the wax in the oils of the mixture is obtained. The waxes may be present in an amount ranging, for example, up to 20% relative to the total weight of the composition, further for example from 0.1% to 15%, and, as a further example, from 1% to 5%.

For the purposes of the invention, the waxes are those generally used in cosmetics and dermatology; such as those of natural origin, for instance beeswax, carnauba wax, candelilla wax, ouricury wax, Japan wax, cork fibre wax, sugar cane wax, paraffin wax, lignite wax, microcrystalline waxes, lanolin wax, montan wax, ozokerites and hydrogenated oils such as hydrogenated jojoba oil, as well as waxes of synthetic origin, for instance polyethylene waxes derived from the polymerization of ethylene, waxes obtained by Fischer-Tropsch synthesis, fatty acid esters and glycerides that are solid at 40° C., for example, at above 55° C., silicone waxes such as alkyl- and alkoxy-poly(di)methylsiloxanes and/or poly(di)methylsiloxane esters that are solid at 40° C. and, for example, at above 55° C.

According to the invention, the melting point values correspond to the melting peak measured by the Differential Scanning Calorimetry method with a temperature rise of 5 or 10° C./min.

The composition according to the invention may be manufactured by the known processes, that are generally used in cosmetics or dermatology. It may be manufactured by the process which comprises heating the at least one structuring polymer at least to its softening point, adding, the at least one pasty fatty substance, and adding, if present, the at least one amphiphilic compound, the at least one coloring agent and the additive(s) thereto and then mixing everything together until a clear, transparent solution is obtained. After reducing the temperature, the volatile solvent(s) is (are) then added to the mixture obtained. The homogeneous mixture obtained can then be cast in a suitable mold such as a lipstick mold or directly into the packaging articles (for example, a case or dish in particular).

An aspect of the invention is also a care, make-up or treatment cosmetic process for keratin materials of human beings, such as superficial body growths, such as keratinous fibers, the skin, the lips, comprising the application to the keratin materials of the composition, for example the cosmetic composition, as defined above.

Another aspect of the invention is the use of the composition of the invention, discussed above, for the manufacture of a physiologically acceptable composition, the combination serving to give the said composition at least one of the following properties: staying power or long wearing properties, a solid appearance, in particular without wax; non-migrating; non-exudation; shear-strength properties; and/or properties of producing a glossy and/or comfortable deposit on keratin materials. In one embodiment, the liquid fatty phase of the composition is essentially structured by an effective amount of at least one structuring polymer and at least one pasty fatty substance.

In a further embodiment, the structuring polymer comprises a polymer skeleton which comprises at least one heteroatom and further comprises at least one of: at least one pendant fatty chain comprising from 12 to 120 carbon atoms bonded to said polymer skeleton via a linking group, and at least one terminal fatty chain comprising from 12 to 120 carbon atoms bonded to said polymer skeleton via a linking group. The at least one pendant fatty chain is optionally functionalized and the at least one terminal fatty chain is optionally functionalized. The percentage of the total number of fatty chains ranges from 40% to 98% relative to the total number of repeating units and fatty chains.

In an even further embodiment, the at least one structuring polymer is a polyamide comprising at least one terminal fatty chain functionalized with an ester comprising a hydrocarbon-based chain having from 10 to 42 carbon atoms.

The invention is illustrated in greater detail in the examples which follow. The amounts are given as percentages by mass.

EXAMPLE 1
Lipstick

| | |
|---|---|
| Uniclear 80 | 18.00% |
| Parleam oil | 26.07% |
| Octyldodecanol | 9.00% |
| Poly(12-hydroxystearic acid) | 2.00% |
| Pigments | 8.66% |
| Lanolin (pasty) | 5.00% |
| Waxes | 3.00% |
| Fillers | 3.00% |
| Phenylated silicone (oil) | 5.00%. |

The Uniclear 80 was dissolved (solubilized) in the parleam oil at 100° C. by virtue of the octyldodecanol and then the pigments and the fillers were added. The waxes and the oils, melted beforehand at 90° C., were added. The combined material was then mixed using a deflocculating turbine (Raynerie) and then cast in lipstick moulds.

The lipstick obtained was glossy, did not migrate and had good hold. This was confirmed by a test on a panel of experts, in comparison with a glossy product of the prior art, Rouge Absolu from Lancôme. This was because, for a comparable gloss and comparable comfort, the lipstick of the invention had much better hold over time and in particular as regards its colour. In addition, it was considered to be glossier than a long-lasting product of the prior art, such as Rouge Magnetic, and more comfortable.

What is claimed is:

1. An anhydrous composition comprising at least one liquid fatty phase which comprises:
(i) at least one structuring polymer comprising:
a polymer skeleton which comprises at least one hydrocarbon-based repeating unit comprising at least one hetero atom; and
(ii) at least one pasty fatty substance.

2. The anhydrous composition according to claim 1, wherein said at least one structuring polymer further comprises at least one of:
at least one terminal fatty chain chosen from alkyl chains and alkenyl chains, wherein said at least one terminal fatty chain is bonded to said polymer skeleton via at least one linking group; and
at least one pendant fatty chain chosen from alkyl chains and alkenyl chains, wherein said at least one pendant fatty chain is bonded to said polymer skeleton via at least one linking group.

3. The anhydrous composition according to claim 2, wherein said alkyl chains and said alkenyl chains each comprise at least four carbon atoms.

4. The anhydrous composition according to claim 3, wherein said alkyl chains and said alkenyl chains each comprise from 8 to 120 carbon atoms.

5. The anhydrous composition according to claim 4, wherein said alkyl chains and said alkenyl chains each comprise from 12 to 68 carbon atoms.

6. The anhydrous composition according to claim 2, wherein said at least one linking group is chosen from single bonds and urea, urethane, thiourea, thiourethane, thioether, thioester, ester, ether, perfluoro, carboxylic acid, hydroxyl, polyol, amide, phosphoric acid, phosphate, carbamate, thiol and amine groups.

7. The anhydrous composition according to claim 6, wherein said at least one linking group is an ester group present in a proportion ranging from 15% to 40% of the total number of all ester and hetero atom groups in the at least one structuring polymer.

8. The anhydrous composition according to claim 7, wherein said at least one linking group is an ester group present in a proportion ranging from 20% to 35% of the total number of all ester and hetero atom groups in the at least one structuring polymer.

9. The anhydrous composition according to claim 6, wherein said at least
one linking group is chosen from urea, ester, and amine groups.

10. The anhydrous composition according to claim 9, wherein said at least one linking group is chosen from ester and amine groups.

11. The anhydrous composition according to claim 2, wherein said at least one terminal fatty chain is functionalized.

12. The anhydrous composition according to claim 2, wherein said at least one pendant fatty chain is functionalized.

13. The anhydrous composition according to claim 2, wherein in said at least one structuring polymer, the percentage of the total number of fatty chains ranges from 40% to 98% relative to the total number of all repeating units and fatty chains in the at least one structuring polymer.

14. The anhydrous composition according to claim 13, wherein in said at least one structuring polymer, the percentage of the total number of fatty chains ranges from 50% to 95% relative to the total number of all repeating units and fatty chains in the at least one structuring polymer.

15. The anhydrous composition according to claim 1, wherein said at least one structuring polymer has a weight-average molecular mass of less than 100,000.

16. The anhydrous composition according to claim 15, wherein said at least one structuring polymer has a weight-average molecular mass of less than 50,000.

17. The anhydrous composition according to claim 16, wherein said at least one structuring polymer has a weight-average molecular mass ranging from 1000 to 30,000.

18. The anhydrous composition according to claim 17, wherein said at least one structuring polymer has a weight-average molecular mass ranging from 2000 to 20,000.

19. The anhydrous composition according to claim 18, wherein said at least one structuring polymer has a weight-average molecular mass ranging from 2000 to 10,000.

20. The anhydrous composition according to claim 1, wherein said at least one hydrocarbon based repeating unit comprises from 2 to 80 carbon atoms.

21. The anhydrous composition according to claim 20, wherein said at least one hydrocarbon based repeating unit comprises from 2 to 60 carbon atoms.

22. The anhydrous composition according to claim 1, wherein said at least one hydrocarbon based repeating unit is chosen from saturated and unsaturated hydrocarbon-based units which are chosen from linear hydrocarbon-based repeating units, branched hydrocarbon-based repeating units and cyclic hydrocarbon-based repeating units.

23. The anhydrous composition according to claim 1, wherein said at least one hetero atom of said at least one hydrocarbon-based repeating unit is chosen from nitrogen, sulphur, and phosphorus.

24. The anhydrous composition according to claim 23, wherein said at least one hetero atom is a nitrogen atom.

25. The anhydrous composition according to claim 23, wherein said at least one hetero atom is combined with at least one atom chosen from oxygen and carbon to form a hetero atom group.

26. The anhydrous composition according to claim 25, wherein said at least one hetero atom group further comprises a carbonyl group.

27. The anhydrous composition according to claim 25, wherein said at least one hetero atom group is chosen from amide groups, carbamate groups, and urea groups.

28. The anhydrous composition according to claim 27, wherein said at least one hetero atom group is an amide group and said polymer skeleton is a polyamide skeleton.

29. The anhydrous composition according to claim 27, wherein said at least one hetero atom group is chosen from carbamate groups and urea groups and said polymer skeleton is chosen from polyurethane skeletons, polyurea skeletons, and polyurethane-polyurea skeletons.

30. The anhydrous composition according to claim 1, wherein said at least one structuring polymer is chosen from polyamide polymers of formula (I): 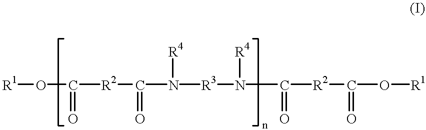
in which:
n is an integer which represents the number of amide units such that the number of ester groups present in said at least one polyamide polymer ranges from 10% to 50% of the total number of all ester groups and all amide groups comprised in said at least one polyamide polymer;
R¹, which are identical or different, are each chosen from alkyl groups comprising at least 4 carbon atoms and alkenyl groups comprising at least 4 carbon atoms;
R², which are identical or different, are each chosen from C₄ to C₄₂ hydrocarbon-based groups with the proviso that at least 50% of all R² are chosen from C₃₀ to C₄₂ hydrocarbon-based groups;
R³, which are identical or different, are each chosen from organic groups comprising atoms chosen from carbon atoms, hydrogen atoms, oxygen atoms and nitrogen atoms with the proviso that R³ comprises at least 2 carbon atoms; and
R⁴, which are identical or different, are each chosen from hydrogen atoms, C₁ to C₁₀ alkyl groups and a direct bond to at least one group chosen from R³ and another R⁴ such that when said at least one group is chosen from another R⁴, the nitrogen atom to which both R³ and R⁴ are bonded forms part of a heterocyclic structure defined in part by R⁴—N—R³, with the proviso that at least 50% of all R⁴ are chosen from hydrogen atoms.

31. The anhydrous composition according to claim 30, wherein in said formula (I), n is an integer ranging from 1 to 5.

32. The anhydrous composition according to claim 31, wherein in said formula (I), n is an integer ranging from 3 to 5.

33. The anhydrous composition according to claim 30, wherein in said formula (I), said alkyl groups of R¹ and said alkenyl groups of R¹ each independently comprise from 4 to 24 carbon atoms.

34. The anhydrous composition according to claim 33, wherein in said formula (I), R¹, which are identical or different, are each chosen from C₁₂ to C₂₂ alkyl groups.

35. The anhydrous composition according to claim 34, wherein in said formula (I), R¹, which are identical or different, are each chosen from C₁₆ to C₂₂ alkyl groups.

36. The anhydrous composition according to claim 30, wherein in said formula (I), R², which are identical or different, are each chosen from C₁₀ to C₄₂ hydrocarbon based groups with the proviso that at least 50% of all R² are chosen from C₃₀ to C₄₂ hydrocarbon based groups.

37. The anhydrous composition according to claim 36, wherein at least 75% of all R², which are identical or different, are chosen from C₃₀ to C₄₂ hydrocarbon based groups.

38. The anhydrous composition according to claim 30, wherein in said formula (I), R³, which can be identical or different, are each chosen from C₂ to C₃6 hydrocarbon-based groups and polyoxyalkylene groups.

39. The anhydrous composition according to claim 38, wherein R³, which can be identical or different, are each chosen from C₂ to C₁₂ hydrocarbon-based groups.

40. The anhydrous composition according to claim 39, wherein in said formula (I), R⁴, which can be identical or different, are each chosen from hydrogen atoms.

41. The anhydrous composition according to claim 30, wherein said at least one polymer of formula (I) is in the form of a mixture of polymers, wherein said mixture optionally also comprises a compound of formula (I) wherein n is equal to zero.

42. The anhydrous composition according to claim 1, wherein said at least one structuring polymer has a softening point greater than 50° C.

43. The anhydrous composition according to claim 42, wherein said at least one structuring polymer has a softening point ranging from 65° C. to 190° C.

44. The anhydrous composition according to claim 43, wherein said at least one structuring polymer has a softening point ranging from 70° C. to 130° C.

45. The anhydrous composition according to claim 44, wherein said at least one structuring polymer has a softening point ranging from 80° C. to 105° C.

46. The anhydrous composition according to claim 1, wherein said at least one structuring polymer is present in the composition in an amount ranging from 0.5% to 80% by weight relative to the total weight of the composition.

47. The anhydrous composition according to claim 46, wherein said at least one structuring polymer is present in the composition in an amount ranging from 2% to 60% by weight relative to the total weight of the composition.

48. The anhydrous composition according to claim 47, wherein said at least one structuring polymer is present in the composition in an amount ranging from 5% to 40% by weight relative to the total weight of the composition.

49. The anhydrous composition according to claim 1, wherein said composition has a hardness ranging from 30 to 300 g.

50. The anhydrous composition according to claim 49, wherein said composition has a hardness ranging from 30 to 250 g.

51. The anhydrous composition according to claim 50, wherein said composition has a hardness ranging from 30 to 200 g.

52. The anhydrous composition according to claim 1, wherein said at least one liquid fatty phase of the composition further comprises at least one oil.

53. The anhydrous composition according to claim 52, wherein said at least one oil is chosen from at least one polar oil and at least one apolar oil.

54. The anhydrous composition according to claim 53, wherein said at least one polar oil is chosen from:
hydrocarbon-based plant oils with a high content of triglycerides comprising fatty acid esters and esters of glycerol in which the fatty acids comprise chains having from 4 to 24 carbon atoms, said chains possibly being chosen from linear and branched, and saturated and unsaturated chains;
synthetic oils or esters of formula R₅COOR₆ in which R₅ is chosen from linear and branched fatty acid residues comprising from 1 to 40 carbon atoms, R₆ is chosen from a hydrocarbon-based chain comprising from 1 to 40 carbon atoms, and R₅+R₆≧10;
synthetic ethers comprising from 10 to 40 carbon atoms;
C₈ to C₂₆ fatty alcohols; and
C₈ to C₂₆ fatty acids.

55. The anhydrous composition according to claim 53, wherein said at least one apolar oil is chosen from:
silicone oils chosen from volatile and non-volatile, linear and cyclic polydimethylsiloxanes that are liquid at room temperature;
polydimethylsiloxanes comprising alkyl or alkoxy groups which are pendant and/or at the end of the silicone chain, the groups each comprising from 2 to 24 carbon atoms;
phenylsilicones; and
hydrocarbons chosen from linear and branched, volatile and non-volatile hydrocarbons of synthetic and mineral origin; and
fluorocarbons chosen from linear and branched, volatile and non-volatile fluorocarbons.

56. The anhydrous composition according to claim 1, wherein said at least one liquid fatty phase further comprises at least one non-volatile oil.

57. The anhydrous composition according to claim 56, wherein said at least one non-volatile oil is chosen from hydrocarbon-based oils of mineral, plant and synthetic origin, synthetic esters and ethers, and silicone oils.

58. The anhydrous composition according to claim 1, wherein said at least one liquid fatty phase is present in an amount ranging from 1% to 99% by weight relative to the total weight of the composition.

59. The anhydrous composition according to claim 58, wherein said at least one liquid fatty phase is present in an amount ranging from 5% to 95.5% by weight relative to the total weight of the composition.

60. The anhydrous composition according to claim 59, wherein said at least one liquid fatty phase is present in an amount ranging from 10% to 80% by weight relative to the total weight of the composition.

61. The anhydrous composition according to claim 60, wherein said at least one liquid fatty phase is present in an amount ranging from 20% to 75% by weight relative to the total weight of the composition.

62. The anhydrous composition according to claim 1, wherein said at least one liquid fatty phase comprises at least one volatile solvent chosen from hydrocarbon-based solvents and silicone solvents optionally comprising at least one group chosen from alkyl and alkoxy groups that are pendant and/or at the end of a silicone chain.

63. The anhydrous composition according to claim 62, wherein said at least one volatile solvent is present in an amount up to 95.5% relative to the total weight of the composition.

64. The anhydrous composition according to claim 63, wherein said at least one volatile solvent is present in an amount ranging from 2% to 75% relative to the total weight of the composition.

65. The anhydrous composition according to claim 64, wherein said at least one volatile solvent is present in an amount ranging from 10% to 45% relative to the total weight of the composition.

66. The anhydrous composition according to claim 1, wherein said at least one pasty fatty substance is chosen from fatty substances having a melting point ranging from 20 to 55° C. and fatty substances having a viscosity at 40° C. ranging from 0.1 to 40 Pa.s.

67. The anhydrous composition according to claim 66, wherein said at least one pasty fatty substance is chosen from fatty substances having a melting point ranging from 25 to 45° C. and fatty substances having a viscosity at 40° C. ranging from 0.5 to 25 Pa.s.

68. The anhydrous composition according to claim 66, wherein said at least one pasty fatty substance is chosen from lanolins, lanolin derivatives, esters of fatty acids, esters of fatty alcohols, arachidyl propionate, polyvinyl laurate, cholesterol esters, polyesters and silicone fatty substances.

69. The anhydrous composition according to claim 68, wherein said lanolin derivatives are chosen from acetylated lanolins, oxypropylenated lanolins and isopropyl lanolate.

70. The anhydrous composition according to claim 68, wherein said cholesterol esters are chosen from triglycerides of plant origin.

71. The anhydrous composition according to claim 68, wherein said polyesters are poly(12-hydroxystearic acid).

72. The anhydrous composition according to claim 68, wherein said silicone fatty substances are chosen from polydimethylsiloxanes (PDMS) having at least one pendant chain chosen from alkyl and alkoxy chains containing from 8 to 24 carbon atoms.

73. The anhydrous composition according to claim 1, wherein said at least one pasty fatty substance is present in a proportion ranging from 0.5% to 60% by weight relative to the total weight of the composition.

74. The anhydrous composition according to claim 1, wherein said at least one pasty fatty substance is present in a proportion ranging from 2% to 45% by weight relative to the total weight of the composition.

75. The anhydrous composition according to claim 1, wherein said at least one pasty fatty substance is present in a proportion ranging from 5% to 30% by weight relative to the total weight of the composition.

76. The anhydrous composition according to claim 1, wherein the composition is in a form chosen from a fluid anhydrous gel, rigid anhydrous gel, fluid simple emulsion, rigid simple emulsion, fluid multiple emulsion, and rigid multiple emulsion.

77. The anhydrous composition according to claim 1, wherein said anhydrous composition is a solid.

78. The anhydrous composition according to claim 77, wherein said anhydrous composition is a solid chosen from molded and poured sticks.

79. The anhydrous composition according to claim 1, further comprising at least one amphiphilic compound that is liquid and non-volatile at room temperature and has a hydrophilic/lipophilic balance value of less than 12.

80. The anhydrous composition according to claim 79, wherein said at least one amphiphilic compound comprises a lipophilic part linked to a polar part, the lipophilic part comprising a carbon-based chain comprising at least 8 carbon atoms.

81. The anhydrous composition according to claim 79, wherein said at least one amphiphilic compound is present in an amount ranging from 0.1% to 35% by weight relative to the total weight of the composition.

82. The anhydrous composition according to claim 81, wherein said at least one amphiphilic compound is present in an amount ranging from 1% to 20% by weight relative to the total weight of the composition.

83. The anhydrous composition according to claim 82, wherein said at least one amphiphilic compound is present in an amount ranging from 1% to 15% by weight relative to the total weight of the composition.

84. The anhydrous composition according to claim 1, further comprising at least one additional additive chosen from antioxidants, essential oils, preservatives, fragrances, fillers, waxes, neutralizing agents, dispersing agents, fat-soluble polymers, cosmetic and dermatological active agents, and an aqueous phase comprising water that is optionally thickened or gelled with an aqueous-phase thickener or gelling agent and optionally water-miscible compounds.

85. The anhydrous composition according to claim 1, further comprising at least one coloring agent.

86. The anhydrous composition according to claim 85, wherein said at least one coloring agent is chosen from lipophilic dyes, hydrophilic dyes, pigments and nacres.

87. The anhydrous composition according to claim 85, wherein said at least one coloring agent is present in a proportion of from 0.01% to 50% relative to the total weight of the composition.

88. The anhydrous composition according to claim 1, wherein said composition is in the form of a rigid gel.

89. The anhydrous composition according to claim 1, wherein said composition is in the form of an anhydrous stick.

90. The anhydrous composition according to claim 1, wherein said composition further comprises at least one wax.

91. The anhydrous composition according to claim 90, wherein said at least one wax is chosen from beeswax, carnauba wax, candelilla wax, ouricury wax, Japan wax, cork fibre wax, sugar cane wax, paraffin wax, lignite wax, microcrystalline waxes, lanolin wax, montan wax, ozokerites and hydrogenated oils, polyethylene waxes, waxes obtained by Fischer-Tropsch synthesis, fatty acid esters and glycerides that are solid at 40° C., and silicone waxes.

92. A composition comprising at least one liquid fatty phase which comprises:
(i) at least one structuring polymer comprising:
a polymer skeleton which comprises at least one hydrocarbon-based repeating unit comprising at least one hetero atom; and
(ii) at least one pasty fatty substance.

93. The composition according to claim 92, wherein said at least one structuring polymer further comprises at least one of:
at least one terminal fatty chain chosen from alkyl chains and alkenyl chains, wherein said at least one terminal fatty chain is bonded to said polymer skeleton via at least one linking group; and
at least one pendant fatty chain chosen from alkyl chains and alkenyl chains, wherein said at least one pendant fatty chain is bonded to said polymer skeleton via at least one linking group.

94. The composition according to claim 93, wherein said alkyl chains and said alkenyl chains each comprise at least four carbon atoms.

95. The composition according to claim 94, wherein said alkyl chains and said alkenyl chains each comprise from 8 to 120 carbon atoms.

96. The composition according to claim 95, wherein said alkyl chains and said alkenyl chains each comprise from 12 to 68 carbon atoms.

97. The composition according to claim 93, wherein said at least one linking group is chosen from single bonds and urea, urethane, thiourea, thiourethane, thioether, thioester, ester, ether, perfluoro, carboxylic acid, hydroxyl, polyol, amide, phosphoric acid, phosphate, carbamate, thiol and amine groups.

98. The composition according to claim 97, wherein said at least one linking group is chosen from urea, ester, and amine groups.

99. The composition according to claim 98, wherein said at least one linking group is chosen from ester and amine groups.

100. The composition according to claim 93, wherein said at least one linking group is an ester group present in a proportion ranging from 15% to 40% of the total number of all ester and hetero atom groups in the at least one structuring polymer.

101. The composition according to claim 100, wherein said at least one linking group is an ester group present in a proportion ranging from 20% to 35% of the total number of all ester and hetero atom groups in the at least one structuring polymer.

102. The composition according to claim 93, wherein said at least one terminal fatty chain is functionalized.

103. The composition according to claim 93, wherein said at least one pendant fatty chain is functionalized.

104. The composition according to claim 93, wherein in said at least one structuring polymer, the percentage of the total number of fatty chains ranges from 40% to 98% relative to the total number of all repeating units and fatty chains in the at least one structuring polymer.

105. The composition according to claim 104, wherein in said at least one structuring polymer, the percentage of the total number of fatty chains ranges from 50% to 95% relative to the total number of all repeating units and fatty chains in the at least one structuring polymer.

106. The composition according to claim 92, wherein said at least one structuring polymer has a weight-average molecular mass of less than 100,000.

107. The composition according to claim 106, wherein said at least one structuring polymer has a weight-average molecular mass of less than 50,000.

108. The composition according to claim 107, wherein said at least one structuring polymer has a weight-average molecular mass ranging from 1000 to 30,000.

109. The composition according to claim 108, wherein said at least one structuring polymer has a weight-average molecular mass ranging from 2000 to 20,000.

110. The composition according to claim 109, wherein said at least one structuring polymer has a weight-average molecular mass ranging from 2000 to 10,000.

111. The composition according to claim 92, wherein said at least one hydrocarbon based repeating unit comprises from 2 to 80 carbon atoms.

112. The composition according to claim 111, wherein said at least one hydrocarbon based repeating unit comprises from 2 to 60 carbon atoms.

113. The composition according to claim 92, wherein said at least one hydrocarbon based repeating unit is chosen from saturated and unsaturated hydrocarbon-based units which are chosen from linear hydrocarbon-based repeating units, branched hydrocarbon-based repeating units and cyclic hydrocarbon-based repeating units.

114. The composition according to claim 92, wherein said at least one hetero atom of said at least one hydrocarbon-based repeating unit is chosen from nitrogen, sulphur, and phosphorus.

115. The composition according to claim 114, wherein said at least one hetero atom is a nitrogen atom.

116. The composition according to claim 114, wherein said at least one hetero atom is combined with at least one atom chosen from oxygen and carbon to form a hetero atom group.

117. The composition according to claim 116, wherein said at least one hetero atom group further comprises a carbonyl group.

118. The composition according to claim 116, wherein said at least one hetero atom group is chosen from amide groups, carbamate groups, and urea groups.

119. The composition according to claim 118, wherein said at least one hetero atom group is an amide group and said polymer skeleton is a polyamide skeleton.

120. The composition according to claim 118, wherein said at least one hetero atom group is chosen from carbamate groups and urea groups and said polymer skeleton is chosen from polyurethane skeletons, polyurea skeletons and polyurethane-polyurea skeletons.

121. The composition according to claim 92, wherein said at least one structuring polymer is chosen from polyamide polymers of formula (I): 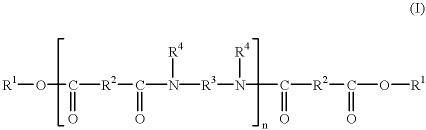
in which:
n is an integer which represents the number of amide units such that the number of ester groups present in said at least one polyamide polymer ranges from 10% to 50% of the total number of all ester groups and all amide groups comprised in said at least one polyamide polymer;
R¹, which are identical or different, are each chosen from alkyl groups comprising at least 4 carbon atoms and alkenyl groups comprising at least 4 carbon atoms;
R², which are identical or different, are each chosen from C₄ to C₄₂ hydrocarbon-based groups with the proviso that at least 50% of all R² are chosen from C₃₀ to C₄₂ hydrocarbon-based groups;
R³, which are identical or different, are each chosen from organic groups comprising atoms chosen from carbon atoms, hydrogen atoms, oxygen atoms and nitrogen atoms with the proviso that R³ comprises at least 2 carbon atoms; and
R⁴, which are identical or different, are each chosen from hydrogen atoms, C₁ to C₁₀ alkyl groups and a direct bond to at least one group chosen from R³ and another R⁴ such that when said at least one group is chosen from another R⁴, the nitrogen atom to which both R³ and R⁴ are bonded forms part of a heterocyclic structure defined in part by R⁴—N—R³, with the proviso that at least 50% of all R⁴ are chosen from hydrogen atoms.

122. The composition according to claim 121, wherein in said formula (I), n is an integer ranging from 1 to 5.

123. The composition according to claim 122, wherein in said formula (I), n is an integer ranging from 3 to 5.

124. The composition according to claim 121, wherein in said formula (I), said alkyl groups of R¹ and said alkenyl groups of R¹ each independently comprise from 4 to 24 carbon atoms.

125. The composition according to claim 124, wherein in said formula (I), R¹, which are identical or different, are each chosen from C₁₂ to C₂₂ alkyl groups.

126. The composition according to claim 125, wherein in said formula (I), R¹, which are identical or different, are each chosen from C₁₆ to C₂₂ alkyl groups.

127. The composition according to claim 121, wherein in said formula (I), R², which are identical or different, are each chosen from C₁₀ to C₄₂ hydrocarbon based groups with the proviso that at least 50% of all R² are chosen from C₃₀ to C₄₂ hydrocarbon based groups.

128. The composition according to claim 127, wherein at least 75% of all R², which are identical or different, are chosen from C₃₀ to C₄₂ hydrocarbon based groups.

129. The composition according to claim 121, wherein in said formula (I), R³, which can be identical or different, are each chosen from C₂ to C₃₆ hydrocarbon-based groups and polyoxyalkylene groups.

130. The composition according to claim 129, wherein R³, which can be identical or different, are each chosen from C₂ to C₁₂ hydrocarbon-based groups.

131. The composition according to claim 130, wherein in said formula (I), R⁴, which can be identical or different, are each chosen from hydrogen atoms.

132. The composition according to claim 121, wherein said at least one polymer of formula (I) is in the form of a mixture of polymers, wherein said mixture optionally also comprises a compound of formula (I) wherein n is equal to zero.

133. The composition according to claim 92, wherein said at least one structuring polymer has a softening point greater than 50° C.

134. The composition according to claim 133, wherein said at least one structuring polymer has a softening point ranging from 65° C. to 190° C.

135. The composition according to claim 134, wherein said at least one structuring polymer has a softening point ranging from 70° C. to 130° C.

136. The composition according to claim 135, wherein said at least one structuring polymer has a softening point ranging from 80° C. to 105° C.

137. The composition according to claim 92 wherein said at least one structuring polymer is present in the composition in an amount ranging from 0.5% to 80% by weight relative to the total weight of the composition.

138. The composition according to claim 137, wherein said at least one structuring polymer is present in the composition in an amount ranging from 2% to 60% by weight relative to the total weight of the composition.

139. The composition according to claim 138, wherein said at least one structuring polymer is present in the composition in an amount ranging from 5% to 40% by weight relative to the total weight of the composition.

140. The composition according to claim 92, wherein said composition has a hardness ranging from 30 to 300 g.

141. The composition according to claim 140, wherein said composition has a hardness ranging from 30 to 250 g.

142. The composition according to claim 141, wherein said composition has a hardness ranging from 30 to 200 g.

143. The composition according to claim 92, wherein said at least one liquid fatty phase of the composition further comprises at least one oil.

144. The composition according to claim 143, wherein said at least one oil is chosen from at least one polar oil and at least one apolar oil.

145. The composition according to claim 144, wherein said at least one polar oil is chosen from:
hydrocarbon-based plant oils with a high content of triglycerides comprising fatty acid esters and esters of glycerol in which the fatty acids comprise chains having from 4 to 24 carbon atoms, said chains possibly being chosen from linear and branched, and saturated and unsaturated chains;
synthetic oils or esters of formula R₅COOR₆ in which R₅ is chosen from linear and branched fatty acid residues comprising from 1 to 40 carbon atoms, R₆ is chosen from a hydrocarbon-based chain comprising from 1 to 40 carbon atoms, and R₅+R₆>10;
synthetic ethers comprising from 10 to 40 carbon atoms;
C₈ to C₂₆ fatty alcohols; and
C₈ to C₂₆ fatty acids.

146. The composition according to claim 144, wherein said at least one apolar oil is chosen from:
silicone oils chosen from volatile and non-volatile, linear and cyclic polydimethylsiloxanes that are liquid at room temperature;
polydimethylsiloxanes comprising alkyl or alkoxy groups which are pendant and/or at the end of the silicone chain, the groups each comprising from 2 to 24 carbon atoms;
phenylsilicones;
hydrocarbons chosen from linear and branched, volatile and non-volatile hydrocarbons of synthetic and mineral origin; and
fluorocarbons chosen from linear and branched, volatile and non-volatile fluorocarbons.

147. The composition according to claim 92, wherein said at least one liquid fatty phase further comprises at least one non-volatile oil.

148. The composition according to claim 147, wherein said at least one non-volatile oil is chosen from hydrocarbon-based oils of mineral, plant and synthetic origin, synthetic esters and ethers, and silicone oils.

149. The composition according to claim 92, wherein said at least one liquid fatty phase is present in an amount ranging from 1% to 99% by weight relative to the total weight of the composition.

150. The composition according to claim 149, wherein said at least one liquid fatty phase is present in an amount ranging from 5% to 95.5% by weight relative to the total weight of the composition.

151. The composition according to claim 150, wherein said at least one liquid fatty phase is present in an amount ranging from 10% to 80% by weight relative to the total weight of the composition.

152. The composition according to claim 151, wherein said at least one liquid fatty phase is present in an amount ranging from 20% to 75% by weight relative to the total weight of the composition.

153. The composition according to claim 92, wherein said at least one liquid fatty phase comprises at least one volatile solvent chosen from hydrocarbon-based solvents and silicone solvents optionally comprising at least one group chosen from alkyl and alkoxy groups that are pendant and/or at the end of a silicone chain.

154. The composition according to claim 153, wherein said at least one volatile solvent is present in an amount up to 95.5% relative to the total weight of the composition.

155. The composition according to claim 154, wherein said at least one volatile solvent is present in an amount ranging from 2% to 75% relative to the total weight of the composition.

156. The composition according to claim 155, wherein said at least one volatile solvent is present in an amount ranging from 10% to 45% relative to the total weight of the composition.

157. The composition according to claim 92, wherein said composition further comprises at least one additional fatty material.

158. The composition according to claim 157, wherein said at least one additional fatty material is chosen from gums, fatty materials pasty at ambient temperature, and resins.

159. The composition according to claim 92, wherein said at least one pasty fatty substance is chosen from fatty substances having a melting point ranging from 20 to 55° C. and fatty substances having a viscosity at 40° C. ranging from 0.1 to 40 Pa.s.

160. The composition according to claim 159, wherein said at least one pasty fatty substance is chosen from fatty substances having a melting point ranging from 25 to 45° C. and fatty substances having a viscosity at 40° C. ranging from 0.5 to 25 Pa.s.

161. The composition according to claim 159, wherein said at least one pasty fatty substance is chosen from lanolins, lanolin derivatives, esters of fatty acids, esters of fatty alcohols, arachidyl propionate, polyvinyl laurate, cholesterol esters, polyesters and silicone fatty substances.

162. The composition according to claim 161, wherein said lanolin derivatives are chosen from acetylated lanolins, oxypropylenated lanolins and isopropyl lanolate.

163. The composition according to claim 161, wherein said cholesterol esters are chosen from triglycerides of plant origin.

164. The composition according to claim 161, wherein said polyesters are poly(12-hydroxystearic acid).

165. The composition according to claim 161, wherein said silicone fatty substances are chosen from polydimethylsiloxanes (PDMS) having at least one pendant chain chosen from alkyl and alkoxy chains containing from 8 to 24 carbon atoms.

166. The composition according to claim 92, wherein said at least one pasty fatty substance is present in a proportion ranging from 0.5% to 60% by weight relative to the total weight of the composition.

167. The composition according to claim 166, wherein said at least one pasty fatty substance is present in a proportion ranging from 2% to 45% by weight relative to the total weight of the composition.

168. The composition according to claim 167, wherein said at least one pasty fatty substance is present in a proportion ranging from 5% to 30% by weight relative to the total weight of the composition.

169. The composition according to claim 92, wherein the composition is in a form chosen from a fluid anhydrous gel, rigid anhydrous gel, fluid simple emulsion, rigid simple emulsion, fluid multiple emulsion, and rigid multiple emulsion.

170. The composition according to claim 92, wherein said composition is a solid.

171. The composition according to claim 170, wherein said composition is a solid chosen from molded and poured sticks.

172. The composition according to claim 92, further comprising at least one amphiphilic compound that is liquid and non-volatile at room temperature and has a hydrophilic/lipophilic balance value of less than 12.

173. The composition according to claim 172, wherein said at least one amphiphilic compound comprises a lipophilic part linked to a polar part, the lipophilic part comprising a carbon-based chain comprising at least 8 carbon atoms.

174. The composition according to claim 172, wherein said at least one amphiphilic compound is present in an amount ranging from 0.1% to 35% by weight relative to the total weight of the composition.

175. The composition according to claim 174, wherein said at least one amphiphilic compound is present in an amount ranging from 1% to 20% by weight relative to the total weight of the composition.

176. The composition according to claim 175, wherein said at least one amphiphilic compound is present in an amount ranging from 1% to 15% by weight relative to the total weight of the composition.

177. The composition according to claim 92, further comprising at least one additional additive chosen from antioxidants, essential oils, preservatives, fragrances, fillers, waxes, neutralizing agents, dispersing agents, fat-soluble polymers, cosmetic and dermatological active agents, and an aqueous phase comprising water that is optionally thickened or gelled with an aqueous-phase thickener or gelling agent and optionally water-miscible compounds.

178. The composition according to claim 92, further comprising at least one coloring agent.

179. The composition according to claim 178, wherein said at least one coloring agent is chosen from lipophilic dyes, hydrophilic dyes, pigments and nacres.

180. The composition according to claim 178, wherein said at least one coloring agent is present in a proportion of from 0.01% to 50% relative to the total weight of the composition.

181. The composition according to claim 92, wherein said composition is in the form of a rigid gel.

182. The composition according to claim 92, wherein said composition is in the form of an anhydrous stick.

183. The composition according to claim 92, wherein said composition further comprises at least one wax.

184. The composition according to claim 183, wherein said at least one wax is chosen from beeswax, carnauba wax, candelilla wax, ouricury wax, Japan wax, cork fibre wax, sugar cane wax, paraffin wax, lignite wax, microcrystalline waxes, lanolin wax, montan wax, ozokerites and hydrogenated oils, polyethylene waxes, waxes obtained by Fischer-Tropsch synthesis, fatty acid esters and glycerides that are solid at 40° C., and silicone waxes.

185. An composition comprising at least one liquid fatty phase which comprises:
(i) at least one structuring polymer, wherein said at least one structuring polymer is at least one polyamide polymer comprising:
a polymer skeleton which comprises at least one amide repeating unit; and
(ii) at least one pasty fatty substance.

186. The composition according to claim 185, wherein said at least one polyamide polymer is chosen from polymers resulting from at least one polycondensation reaction between at least one acid chosen from dicarboxylic acids comprising at least 32 carbon atoms and at least one amine chosen from diamines comprising at least 2 carbon atoms and triamines comprising at least 2 carbon atoms.

187. The composition according to claim 186, wherein said dicarboxylic acids comprise from 32 to 44 carbon atoms and said at least one amine comprises from 2 to 36 carbon atoms.

188. The composition according to claim 187, wherein said dicarboxylic acids are chosen from dimers of at least one fatty acid comprising at least 16 carbon atoms.

189. The composition according to claim 188, wherein said at least one fatty acid is chosen from oleic acid, linoleic acid and linolenic acid.

190. The composition according to claim 186, wherein said diamines are chosen from ethylenediamine, hexylenediamine, hexamethylenediamine, and phenylenediamine and said triamines are chosen from ethylenetriamine.

191. The composition according to claim 185, wherein said at least one polyamide polymer is chosen from polymers comprising at least one terminal carboxylic acid group.

192. The composition according to claim 191, wherein said at least one terminal carboxylic acid group is esterified with at least one alcohol chosen from monoalcohols comprising at least 4 carbon atoms.

193. The composition according to claim 185, wherein said at least one polyamide polymer is chosen from:
polymers chosen from mixtures of copolymers derived from monomers of (i) C₃₆ diacids and (ii) ethylenediamine, and having a weight-average molecular mass of about 6000;
polyamide polymers resulting from the condensation of at least one aliphatic dicarboxylic acid and at least one diamine, the carbonyl and amine groups being condensed via an amide bond; and
polyamide resins from vegetable sources.

194. The composition according to claim 185, further comprising at least one amphiphilic compound that is liquid and non-volatile at room temperature and has a hydrophilic/lipophilic balance value of less than 12.

195. The composition according to claim 194, wherein said at least one amphiphilic compound comprises a lipophilic part linked to a polar part, the lipophilic part comprising a carbon-based chain comprising at least 8 carbon atoms.

196. The composition according to claim 194, wherein said at least one amphiphilic compound is present in an amount ranging from 0.1% to 35% by weight relative to the total weight of the composition.

197. The composition according to claim 196, wherein said at least one amphiphilic compound is present in an amount ranging from 1% to 20% by weight relative to the total weight of the composition.

198. The composition according to claim 197, wherein said at least one amphiphilic compound is present in an amount ranging from 1% to 15% by weight relative to the total weight of the composition.

199. The composition according to claim 185, wherein said at least one liquid fatty phase of the composition further comprises at least one oil.

200. The composition according to claim 185, further comprising at least one additional additive chosen from antioxidants, essential oils, preservatives, fragrances, fillers, waxes, neutralizing agents, dispersing agents, fat-soluble polymers, cosmetic and dermatological active agents, and an aqueous phase comprising water that is optionally thickened or gelled with an aqueous-phase thickener or gelling agent and optionally water-miscible compounds.

201. The composition according to claim 185, further comprising at least one coloring agent.

202. The composition according to claim 201, wherein said at least one coloring agent is chosen from lipophilic dyes, hydrophilic dyes, pigments and nacres.

203. The composition according to claim 201, wherein said at least one coloring agent is present in a proportion of from 0.01% to 50% relative to the total weight of the composition.

204. The composition according to claim 185, wherein said composition is in the form of a rigid gel.

205. The composition according to claim 185, wherein said composition is in the form of an anhydrous stick.

206. The composition according to claim 185, wherein said composition further comprises at least one wax.

207. The composition according to claim 206, wherein said at least one wax is chosen from beeswax, carnauba wax, candelilla wax, ouricury wax, Japan wax, cork fibre wax, sugar cane wax, paraffin wax, lignite wax, microcrystalline waxes, lanolin wax, montan wax, ozokerites and hydrogenated oils, polyethylene waxes, waxes obtained by Fischer-Tropsch synthesis, fatty acid esters and glycerides that are solid at 40° C., and silicone waxes.

208. The composition according to claim 185, wherein said at least one pasty fatty substance is chosen from fatty substances having a melting point ranging from 20 to 55° C. and fatty substances having a viscosity at 40° C. ranging from 0.1 to 40 Pa.s.

209. The composition according to claim 208, wherein said at least one pasty fatty substance is chosen from fatty substances having a melting point ranging from 25 to 45° C. and fatty substances having a viscosity at 40° C. ranging from 0.5 to 25 Pa.s.

210. The composition according to claim 185, wherein said at least one pasty fatty substance is chosen from lanolins, lanolin derivatives, esters of fatty acids, esters of fatty alcohols, arachidyl propionate, polyvinyl laurate, cholesterol esters, polyesters and silicone fatty substances.

211. The composition according to claim 210, wherein said lanolin derivatives are chosen from acetylated lanolins, oxypropylenated lanolins and isopropyl lanolate.

212. The composition according to claim 210, wherein said cholesterol esters are chosen from triglycerides of plant origin.

213. The composition according to claim 210, wherein said polyesters are poly(12-hydroxystearic acid).

214. The composition according to claim 210, wherein said silicone fatty substances are chosen from polydimethylsiloxanes (PDMS) having at least one pendant chain chosen from alkyl and alkoxy chains containing from 8 to 24 carbon atoms.

215. The composition according to claim 185, wherein said at least one pasty fatty substance is present in a proportion ranging from 0.5% to 60% by weight relative to the total weight of the composition.

216. The composition according to claim 185, wherein said at least one pasty fatty substance is present in a proportion ranging from 2% to 45% by weight relative to the total weight of the composition.

217. The composition according to claim 185, wherein said at least one pasty fatty substance is present in a proportion ranging from 5% to 30% by weight relative to the total weight of the composition.

218. A mascara, an eyeliner, a foundation, a lipstick, a make-up-removing product, a make-up product for the body, a nail composition, an eyeshadow, a face powder, a concealer product, a shampoo, a conditioner, an antisun product or a care product for the lips, face, body, or hair comprising a composition comprising at least one liquid fatty phase in said mascara, eyeliner, foundation, lipstick, blusher, make-up-removing product, make-up product for the body, nail composition, eyeshadow, face powder, concealer product, shampoo, conditioner, antisun product or care product for the lips, face, body, or hair which comprises:
(i) at least one structuring polymer comprising:
a polymer skeleton which comprises at least one hydrocarbon-based repeating unit comprising at least one hetero atom; and
(ii) at least one pasty fatty substance.

219. A deodorant product or a care product for the skin or body comprising an composition comprising at least one liquid fatty phase in said product which comprises:
(i) at least one structuring polymer comprising:
a polymer skeleton which comprises at least one hydrocarbon-based repeating unit comprising at least one hetero atom; and
(ii) at least one pasty fatty substance.

220. A care and/or treatment and/or make-up composition for keratin materials comprising an anhydrous composition comprising at least one liquid fatty phase which comprises:
(i) at least one structuring polymer comprising:
a polymer skeleton which comprises at least one hydrocarbon-based repeating unit comprising at least one hetero atom; and
(ii) at least one pasty fatty substance.

221. A care and/or treatment and/or make-up composition for keratinous fibers, lips or skin comprising at least one liquid fatty phase in said care and/or treatment and/or make-up composition for keratinous fibers, lips or skin which comprises:
(i) at least one structuring polymer comprising:
a polymer skeleton which comprises at least one hydrocarbon-based repeating unit comprising at least one hetero atom; and
(ii) at least one pasty fatty substance.

222. A lipstick composition in stick form comprising at least one continuous liquid fatty phase, at least one pasty fatty substance, and at least one structuring polymer having a weight-average molecular mass of less than 100,000, said at least one structuring polymer comprising:
a polymer skeleton which comprises at least one hydrocarbon-based repeating unit comprising at least one hetero atom.

223. A method for care, make-up or treatment of keratin materials comprising applying to said keratin materials a composition comprising at least one liquid fatty phase which comprises:
(i) at least one structuring polymer comprising:
a polymer skeleton which comprises at least one hydrocarbon-based repeating unit comprising at least one hetero atom; and
(ii) at least one pasty fatty substance.

224. A method for care, make-up or treatment of keratinous fibers, lips, or skin comprising applying to said keratinous fibers, lips, or skin a composition comprising at least one liquid fatty phase which comprises:
(i) at least one structuring polymer comprising:
a polymer skeleton which comprises at least one hydrocarbon-based repeating unit comprising at least one hetero atom; and
(ii) at least one pasty fatty substance.

225. A method for providing a composition having at least one property chosen from a solid appearance, non-exudation, shear-strength, gloss, and comfortable deposit on keratin materials chosen from lips, skin, and keratinous fibers, comprising including in said composition at least one liquid fatty phase which comprises:
(i) at least one structuring polymer comprising:
a polymer skeleton which comprises at least one hydrocarbon-based repeating unit comprising at least one hetero atom; and
(ii) at least one pasty fatty substance.

226. A structured composition comprising at least one liquid fatty phase structured with at least one structuring polymer, wherein said at least one structuring polymer is at least one polyamide polymer comprising a polymer skeleton which comprises at least one amide repeating unit, wherein the at least one structuring polymer further comprises at least one chain chosen from (i) terminal fatty chains, optionally functionalized, chosen from alkyl and alkenyl chains, bonded to the polymer skeleton via at least one linking group chosen from amides, ureas, and esters, and (ii) pendant fatty chains, optionally functionalized, chosen from alkyl and alkenyl chains, bonded to the polymer skeleton via at least one linking group chosen from amides, ureas, and esters, wherein when said at least one linking group is chosen from esters, said at least one terminal fatty chain is chosen from branched alkyl groups, and further comprising at least one pasty fatty substance.

227. A structured composition comprising at least one liquid fatty phase structured with at least one structuring polymer comprising a polymer skeleton comprising at least one hydrocarbon-based repeating unit comprising at least one hetero atom, wherein the at least one structuring polymer further comprises at least one chain chosen from (i) terminal fatty chains, optionally functionalized, chosen from alkyl and alkenyl chains, bonded to the polymer skeleton via at least one linking group chosen from amides, ureas, and esters, and (ii) pendant fatty chains, optionally functionalized, chosen from alkyl and alkenyl chains, bonded to the polymer skeleton via at least one linking group chosen from amides, ureas, and esters, wherein when said at least one linking group is chosen from esters, said at least one terminal fatty chain is chosen from branched alkyl groups, and further comprising at least one pasty fatty substance.

228. A make up or care or treatment composition for the skin, the lips, or keratinous fibers comprising a structured composition comprising at least one liquid fatty phase structured with at least one structuring polymer comprising a polymer skeleton comprising at least one hydrocarbon-based repeating unit comprising at least one hetero atom, at least one pasty fatty substance, and at least one coloring agent.

229. A method of making up or caring for skin, lips, or keratinous fibers comprising applying to said skin, lips, or keratinous fibers a structured composition comprising at least one liquid fatty phase structured with at least one structuring polymer comprising a polymer skeleton comprising at least one hydrocarbon-based repeating unit comprising at least one hetero atom and at least one pasty fatty substance.

230. A composition comprising at least one liquid fatty phase which comprises:
(i) at least one structuring polymer comprising
a polymer skeleton which comprises at least one hydrocarbon-based repeating unit comprising at least one hetero atom with the proviso that said at least one hetero atom is not nitrogen; and
(ii) at least one pasty fatty substance.

231. The anhydrous composition according to claim 79, wherein said hydrophilic/lipophilic balance value is less than 8.

232. The anhydrous composition according to claim 231, wherein said hydrophilic/lipophilic balance value ranges from 1 to 7.

233. The anhydrous composition according to claim 232, wherein hydrophilic/lipophilic balance value ranges from 1 to 5.

234. The anhydrous composition according to claim 79, wherein said at least one amphiphilic compound is chosen from hydroxystearates of glycerol, oleates of glycerol, isostearates of glycerol, hydroxystearates of sorbitan, oleates of sorbitan, isostearates of sorbitan, hydroxystearates of methylglucose, oleates of methylglucose, isostearates of methylglucose, and octyldodecanol.

235. The composition according to claim 172, wherein said hydrophilic/lipophilic balance value is less than 8.

236. The composition according to claim 235, wherein said hydrophilic/lipophilic balance value ranges from 1 to 7.

237. The composition according to claim 236, wherein hydrophilic/lipophilic balance value ranges from 1 to 5.

238. The composition according to claim 172, wherein said at least one amphiphilic compound is chosen from hydroxystearates of glycerol, oleates of glycerol, isostearates of glycerol, hydroxystearates of sorbitan, oleates of sorbitan, isostearates of sorbitan, hydroxystearates of methylglucose, oleates of methylglucose, isostearates of methylglucose, and octyldodecanol.

239. The composition according to claim 194, wherein said hydrophilic/lipophilic balance value is less than 8.

240. The composition according to claim 239, wherein said hydrophilic/lipophilic balance value ranges from 1 to 7.

241. The composition according to claim 240, wherein hydrophilic/lipophilic balance value ranges from 1 to 5.

242. The composition according to claim 194, wherein said at least one amphiphilic compound is chosen from hydroxystearates of glycerol, oleates of glycerol, isostearates of glycerol, hydroxystearates of sorbitan, oleates of sorbitan, isostearates of sorbitan, hydroxystearates of methylglucose, oleates of methylglucose, isostearates of methylglucose, and octyidodecanol.

243. The anhydrous composition according to claim 80, wherein said carbon-based chain comprises from 16 to 32 carbon atoms.

244. The anhydrous composition according to claim 243, wherein said carbon-based chain comprises from 18 to 28 carbon atoms.

245. The composition according to claim 173, wherein said carbon-based chain comprises from 16 to 32 carbon atoms.

246. The composition according to claim 245, wherein said carbon-based chain comprises from 18 to 28 carbon atoms.

247. The composition according to claim 195, wherein said carbon-based chain comprises from 16 to 32 carbon atoms.

248. The composition according to claim 247, wherein said carbon-based chain comprises from 18 to 28 carbon atoms.

249. The anhydrous composition according to claim 79, wherein said at least one amphiphilic compound comprises a lipophilic part linked to a polar part, wherein the polar part is a residue of a compound chosen from alcohols and polyols comprising from 1 to 12 hydroxyl groups, and polyoxyalkylenes comprising at least 2 oxyalkylene units, from 0 to 20 oxypropylene units, and from 0 to 20 oxyethylene units.

250. The composition according to claim 172, wherein said at least one amphiphilic compound comprises a lipophilic part linked to a polar part, wherein the polar part is a residue of a compound chosen from
alcohols and polyols comprising from 1 to 12 hydroxyl groups, and
polyoxyalkylenes comprising at least 2 oxyalkylene units, from 0 to 20 oxypropylene units, and from 0 to 20 oxyethylene units.

251. The anhydrous composition according to claim 194, wherein said at least one amphiphilic compound comprises a lipophilic part linked to a polar part, wherein the polar part is a residue of a compound chosen from alcohols and polyols comprising from 1 to 12 hydroxyl groups, and polyoxyalkylenes comprising at least 2 oxyalkylene units, from 0 to 20 oxypropylene units, and from 0 to 20 oxyethylene units.

252. The anhydrous composition according to claim 53, wherein said at least one apolar oil is squalane.

253. The composition according to claim 144, wherein said at least one apolar oil is squalane.

254. The anhydrous composition according to claim 68, wherein said esters of fatty acids are chosen from esters of fatty acids comprising from 20 to 65 carbon atoms.

255. The anhydrous composition according to claim 68, wherein said esters of fatty alcohols are chosen from esters of fatty alcohols comprising from 20 to 65 carbon atoms.

256. The composition according to claim 161, wherein said esters of fatty acids are chosen from esters of fatty acids comprising from 20 to 65 carbon atoms.

257. The composition according to claim 161, wherein said esters of fatty alcohols are chosen from esters of fatty alcohols comprising from 20 to 65 carbon atoms.

258. The anhydrous composition according to claim 210, wherein said esters of fatty acids are chosen from esters of fatty acids comprising from 20 to 65 carbon atoms.

259. The anhydrous composition according to claim 210, wherein said esters of fatty alcohols are chosen from esters of fatty alcohols comprising from 20 to 65 carbon atoms.

260. The anhydrous composition according to claim 17, wherein said at least one structuring polymer has a weight-average molecular mass ranging from 1000 to 10,000.

261. The anhydrous composition according to claim 19, wherein said at least one structuring polymer has a weight-average molecular mass ranging from 2000 to 8,000.

262. The composition according to claim 108, wherein said at least one structuring polymer has a weight-average molecular mass ranging from 1000 to 10,000.

263. The composition according to claim 110, wherein said at least one structuring polymer has a weight-average molecular mass ranging from 2000 to 8,000.

264. The anhydrous composition according to claim 1, wherein said polymer skeleton further comprises at least one repeating unit chosen from silicone units and oxyalkylene units, the at least one repeating unit being between the hydrocarbon-based repeating units.

265. The anhydrous composition according to claim 264, wherein said silicone unit forms an organopolysiloxane backbone.

266. The composition according to claim 92, wherein said polymer skeleton further comprises at least one repeating unit chosen from silicone units and oxyalkylene units, the at least one repeating unit being between the hydrocarbon-based repeating units.

267. The composition according to claim 266, wherein said silicone unit forms an organopolysiloxane backbone.

268. The anhydrous composition according to claim 48, wherein said at least one structuring polymer is present in the composition in an amount ranging from 5% to 25% by weight relative to the total weight of the composition.

269. The composition according to claim 139, wherein said at least one structuring polymer is present in the composition in an amount ranging from 5% to 25% by weight relative to the total weight of the composition.

270. The anhydrous composition according to claim 51, wherein said composition has a hardness ranging from 30 to 150 g.

271. The anhydrous composition according to claim 270, wherein said composition has a hardness ranging from 30 to 120 g.

272. The anhydrous composition according to claim 271, wherein said composition has a hardness ranging from 30 to 50 g.

273. The composition according to claim 142, wherein said composition has a hardness ranging from 30 to 150 g.

274. The composition according to claim 273, wherein said composition has a hardness ranging from 30 to 120 g.

275. The composition according to claim 274, wherein said composition has a hardness ranging from 30 to 50 g.

276. The anhydrous composition according to claim 87, wherein said at least one coloring agent is present in a proportion of from 0.5% to 40% relative to the total weight of the composition.

277. The anhydrous composition according to claim 276, wherein said at least one coloring agent is present in a proportion of from 5% to 30% relative to the total weight of the composition.

278. The anhydrous composition according to claim 277, wherein said at least one coloring agent is present in a proportion of from 5% to 25% relative to the total weight of the composition.

279. The anhydrous composition according to claim 180, wherein said at least one coloring agent is present in a proportion of from 0.5% to 40% relative to the total weight of the composition.

280. The anhydrous composition according to claim 279, wherein said at least one coloring agent is present in a proportion of from 5% to 30% relative to the total weight of the composition.

281. The anhydrous composition according to claim 280, wherein said at least one coloring agent is present in a proportion of from 5% to 25% relative to the total weight of the composition.

282. A method for conferring long wearing properties on a composition comprising:
(a) at least one structuring polymer comprising:
a polymer skeleton which comprises at least one hydrocarbon-based repeating unit comprising at least one hetero atom and at least one of:
at least one terminal fatty chain chosen from alkyl chains and alkenyl chains, optionally functionalized, wherein said at least one terminal fatty chain is bonded to said polymer skeleton via at least one linking group and wherein said at least one terminal fatty chain comprises from 12 to 120 carbon atoms; and
at least one pendant fatty chain chosen from alkyl chains and alkenyl chains, optionally functionalized, wherein said at least one pendant fatty chain is bonded to said polymer skeleton via at least one linking group and wherein said at least one terminal fatty chain comprises from 12 to 120 carbon atoms,
wherein in said at least one structuring polymer, the percentage of the total number of fatty chains ranges from 40% to 98% relative to the total number of all repeating units and fatty chains in the at least one structuring polymer; and
(b) at least one pasty fatty substance.

283. The method according to claim 282, wherein said at least one structuring polymer is a polyamide comprising at least one terminal fatty chain functionalized with an ester comprising a hydrocarbon-based chain having from 10 to 42 carbon atoms.

284. A method according to claim 282, further comprising at least one amphiphilic compound which is liquid at room temperature and which has an HLB value of less than 12.

285. A method according to claim 284, wherein said at least one amphiphilic compound has an HLB value of less than 8.

286. A method according to claim 284, wherein said at least one amphiphilic compound has an HLB value ranging from 1 to 7.

287. A method according to claim 284, wherein said at least one amphiphilic compound has an HLB value ranging from 1 to 5.
